(19) United States
(12) Patent Application Publication
(10) Pub. No.: US 2010/0070036 A1
(43) Pub. Date: Mar. 18, 2010

(54) EXTENDABLE INTERVERTEBRAL DISC PROSTHESIS SYSTEM

(76) Inventor: Dante Implicito, Franklin Lakes, NJ (US)

Correspondence Address: KENYON & KENYON LLP, 1500 K STREET N.W., SUITE 700, WASHINGTON, DC, 20005, US

(21) Appl. No.: 12/233,378

(22) Filed: Sep. 18, 2008

(51) Int. Cl. A61F 2/44; A61M 29/00

(52) U.S. Cl. 623/17.16; 623/17.11; 623/17.15; 606/191

(57) ABSTRACT

The present invention generally relates to an extendable intervertebral disc prosthesis system and assemblies thereof. A prosthesis system of certain embodiments includes a superior and inferior endplate assembly as well as an inner core disposed therebetween. Each endplate assembly includes two plates that slide relative to one another longitudinally such that the endplate assembly moves from an unextended position having a first width to an extended position having a second width greater than the first width. The present invention also provides a telescoping intervertebral disc prosthesis system. Additionally, the present invention provides methods for implanting extendable intervertebral disc prosthesis systems and their assemblies. The present invention also provides a kit for implanting an extendable intervertebral disc prosthesis system, including an insertion tool and an extendable intervertebral disc prosthesis system.

### TECHNICAL FIELD

The present invention generally relates to prosthesis systems, assemblies, and methods for replacing a natural intervertebral disc. More specifically, the present invention relates to extendable intervertebral disc prosthesis systems.

### BACKGROUND

The intervertebral disc provides a mechanical cushion between adjacent vertebral segments of the spinal column and also maintains the proper anatomical separation between two adjacent vertebrae. This separation is necessary for allowing both afferent and efferent nerves to respectively exit and enter the spinal column. The disc also plays a key role in maintaining flexibility in the spinal column. In some instances, genetic or developmental irregularities, trauma, chronic stress, or degenerative disease can result in spinal pathologies necessitating removal of an intervertebral disc.

One option after removal of the intervertebral disc is completely replacing the natural disc with an artificial disc prosthesis. There are several designs of such total disc replacement prostheses, both for lumbar and cervical discs. Many of these prostheses are impacted into the intervertebral disc space as a single component and require a rather invasive surgical procedure to implant, due to the numerous sensitive organs surrounding the spine (such as the dural tube). Other disc prostheses may be assembled intradiscally, but these devices usually still require relatively invasive surgical procedures to impact their large endplates or, alternatively, utilize endplates having too small of a superior and inferior surface area to achieve a desirable interface with adjacent vertebral endplates.

In addition, one of the drawbacks to current intervertebral disc prostheses is their inability to be inserted via a posterior approach. This restriction forces surgeons to use an anterior or lateral approach to insert the prosthesis. While anterior and lateral methods are not always undesirable, having the ability for posterior insertion is a key feature sought by surgeons in intervertebral disc replacements.

Accordingly, in order to reduce highly invasive and difficult surgical procedures, but still achieve sufficient interface with adjacent vertebral endplates, there is a need for a different type of intervertebral disc prosthesis that can be implanted in a less invasive manner and that is capable of posterior insertion.

### SUMMARY

The present invention generally relates to extendable intervertebral disc prosthesis systems, assemblies, and methods of use. In certain embodiments, the present invention provides an extendable intervertebral disc prosthesis endplate assembly having a longitudinal axis and comprising a slotted plate and an engagement plate. The slotted plate of these embodiments includes a body (having an outer surface, an inner surface, a posterior surface, and an anterior surface) and at least one slot defined by at least the inner surface of the slotted plate's body. The engagement plate of these embodiments includes a body (having an outer surface, an inner surface, a posterior surface, an anterior surface, and a medial wall), a lip (having a contact surface) extending from the medial wall, and at least one engagement member extending from the contact surface of the lip. When the at least one slot receives the at least one engagement member, the slotted plate slides relative to the engagement plate (or vice versa) along the assembly's longitudinal axis. As the plates slide apart from one another, the assembly moves from an unextended position—having a first width—to an extended position—having a second width that is greater than the first width.

The present invention additionally provides an extendable intervertebral disc prosthesis system, comprising a superior endplate assembly, an inferior endplate assembly, and an inner core. The endplate assemblies of these embodiments each have a longitudinal axis and comprise a slotted plate and an engagement plate. The slotted plate of these embodiments includes a body (having an outer surface, an inner surface, a posterior surface, and an anterior surface) and at least one slot defined by at least the inner surface of the slotted plate's body. The engagement plate of these embodiments includes a body (having an outer surface, an inner surface, a posterior surface, an anterior surface, and a medial wall), a lip (having a contact surface) extending from the medial wall, and at least one engagement member extending from the contact surface of the lip. When the at least one slot receives the at least one engagement member, the slotted plate slides relative to the engagement plate (or vice versa) along the assembly's longitudinal axis. As the plates slide apart from one another, the assembly moves from an unextended position—having a first width—to an extended position—having a second width that is greater than the first width. Additionally, either the slotted plate, the engagement plate, or both further includes an extending portion in fluid communication with the inner surface of the slotted plate, the engagement plate, or both. In an applied position of the assembly, the extending portion of certain embodiments projects in the inferior direction (these embodiments are hereinafter referred to as “superior extendable intervertebral disc prosthesis endplate assembly” or “superior endplate assembly”). Alternatively, in an applied position of the assembly, the extending portion of certain other embodiments projects in the superior direction (these embodiments are hereinafter referred to as “inferior extendable intervertebral disc prosthesis endplate assembly” or “inferior endplate assembly”). The inner core comprises a core body and is configured to be slidably received by the extending portions of the superior and inferior endplate assemblies, thereby holding each endplate assembly in the extended position.

The present invention also provides another extendable intervertebral disc prosthesis system, comprising a superior endplate assembly, an inferior endplate assembly, and an inner core. The superior and inferior endplate assemblies of these embodiments each have a longitudinal axis and comprise a slotted plate and an engagement plate. The slotted plate includes a body (having an outer surface, an inner surface, a posterior surface, and an anterior surface), at least one slot defined by at least the inner surface of the slotted plate's body, and a hook portion defining a groove extending along at least a portion of the space between the posterior and anterior surfaces of the slotted plate's body. The engagement plate includes a body (having an outer surface, an inner surface, a posterior surface, an anterior surface, and a medial wall), a lip (having a contact surface) extending from the medial wall, at least one engagement member extending from the contact surface of the lip, and a hook portion defining a groove extending along at least a portion of the space between the posterior and anterior surfaces of the engagement plate's body. For each endplate assembly, when the at least one slot receives the at least one engagement member, the slotted plate slides relative to the engagement plate (or vice versa) along the assembly's longitudinal axis. As the plates slide apart from one another, the assembly moves from an unextended position—having a first width—to an extended position—having a second width that is greater than the first width. The inner core of these embodiments includes a core body and at least one projection extending from a surface of the inner core's body. The at least one projection of the inner core is configured to be slidably received by the grooves of the slotted plates and the engagement plates. When the inner core of these embodiments slides between the superior and inferior endplate assemblies, the superior and inferior endplate assemblies are held in the extended position, and each endplate assembly is interlocked to the inner core.

The present invention further provides a telescoping intervertebral disc prosthesis system, comprising a superior endplate assembly, an inferior endplate assembly, and an inner core. Each endplate assembly of these embodiments has a longitudinal axis and comprises an inner member and an outer member. The inner members each include a body (having a cross-sectional profile, an outer surface, an inner surface, a posterior surface, and an anterior surface), and a hook portion defining a groove extending along at least a portion of the space between the posterior surface of the body of the inner member and the anterior surface of the body of the inner member. The outer members each include a body (having an outer surface, an inner surface, a posterior surface, and an anterior surface), a longitudinal channel defined by a space large enough to receive the cross-sectional profile of the inner member, and a hook portion defining a groove extending along at least a portion of the space between the posterior surface of the body of the outer member and the anterior surface of the body of the outer member. In each endplate assembly, when the inner member slides within the longitudinal channel of the outer member, the assembly moves from an unextended position having a first width to an extended position having a second width greater than the first width. The inner core includes a core body and at least one projection extending from a surface of the core body. The at least one projection is configured to be slidably received by at least one of the grooves of the superior or inferior endplate assemblies. When the inner core slides between the superior and inferior endplate assemblies, the endplate assemblies are each held in the extended position and are each interlocked to the inner core.

The present invention additionally provides a method for surgically implanting an extendable intervertebral disc prosthesis in an intervertebral disc space. The method comprises removing a patient's natural intervertebral disc from its intervertebral disc space. A superior endplate assembly and an inferior endplate assembly each capable of assuming an unextended position and an extended position are provided. Both the superior and inferior endplate assemblies in the unextended position are simultaneously inserted in the excised intervertebral disc space. After being positioned in the intervertebral disc space, the endplate assemblies are simultaneously distracted to the extended position. An inner core is then implanted between the superior and inferior endplate assemblies.

The present invention additionally provides a method for surgically implanting an extendable intervertebral disc prosthesis in an intervertebral disc space. The method comprises removing a patient's natural intervertebral disc from an intervertebral disc space. A superior endplate assembly and an inferior endplate assembly each capable of assuming an unextended position and an extended position are provided. Either the superior or the inferior endplate assembly in the unextended position is inserted in the excised intervertebral disc space. After being positioned in the intervertebral disc space, the endplate assembly is distracted to the extended position. The other of the superior or inferior endplate assembly in the unextended position is then inserted in the excised disc space, and this endplate assembly is distracted to the extended position. An inner core is then implanted between the superior and inferior endplate assemblies.

The present invention further provides a kit for replacing a natural intervertebral disc with an extendable intervertebral disc prosthesis system. In certain embodiments, the kit includes an extendable intervertebral disc prosthesis and an insertion tool. In certain embodiments, the insertion tool comprises a plurality of insertion fingers (including a left superior insertion finger, a right superior insertion finger, a left inferior insertion finger, and a right inferior insertion finger), means for laterally distracting the left insertion fingers from the right insertion fingers, and a pair of handles pivotally connected to the means for laterally distracting. The handles are capable of distracting, in the superior-inferior direction, the superior insertion fingers from the inferior insertion fingers, and the plurality of fingers is connected (either directly or indirectly) to the means for laterally distracting.

The present invention also provides a kit for replacing a natural intervertebral disc prosthesis with an extendable intervertebral disc prosthesis. The kit includes an extendable intervertebral disc prosthesis and an insertion tool comprising a superior track, an inferior track, a left superior insertion finger, a right superior insertion finger, a left inferior insertion finger, and a right inferior insertion finger. The superior insertion fingers are slidably connected to the superior track such that the superior insertion fingers are capable of moving laterally along the superior track, while the inferior insertion fingers are slidably connected to the inferior track such that the inferior insertion fingers are capable of moving laterally along the inferior track. An insertion tool of these embodiments also includes a pair of handles pivotally connected to the superior and inferior tracks, wherein the handles are capable of distracting, in the superior-inferior direction, the superior insertion fingers from the inferior insertion fingers.

The invention may be embodied in numerous devices and through numerous methods and systems. The following detailed description, taken in conjunction with the annexed drawings, discloses examples of the invention. Other embodiments, which incorporate some, all or more of the features as taught herein, are also possible.

### BRIEF DESCRIPTION OF THE DRAWINGS

Referring to the drawings, which form a part of this disclosure:

FIG. 1A is an isometric view of an extendable intervertebral disc prosthesis endplate assembly in accordance with certain embodiments of the present invention in the extended position;

FIG. 1B is an isometric view of a slotted plate of certain embodiments of the present invention;

FIG. 1C is an isometric view of an engagement plate of certain embodiments of the present invention;

FIG. 1D is an isometric view of an extendable intervertebral disc prosthesis endplate assembly in accordance with certain embodiments of the present invention in the unextended position;

FIG. 2A is an isometric view of a superior extendable intervertebral disc prosthesis endplate assembly in accordance with certain embodiments of the present invention in the extended position;

FIG. 2B is an isometric view of a slotted plate of certain embodiments of the present invention;

FIG. 2C is an isometric view of an engagement plate of certain embodiments of the present invention;

FIG. 3A is an isometric view of an inferior extendable intervertebral disc prosthesis endplate assembly in accordance with certain embodiments of the present invention;

FIG. 3B is an isometric view of the slotted plate of the inferior endplate assembly of FIG. 3A;

FIG. 3C is an isometric view of the engagement plate of the inferior endplate assembly of FIG. 3A;

FIG. 4A is an isometric view of an extendable intervertebral disc prosthesis system in accordance with certain embodiments of the present invention having hook portions shown in relation to an inner core;

FIG. 4B is an isometric view of the prosthesis system of 4A, showing an inner core partially disposed between superior and inferior endplate assemblies;

FIG. 4C is an isometric view of the prosthesis system of 4A in an applied position, showing an inner core fully disposed between superior and inferior endplate assemblies;

FIG. 5A is an isometric view of an extendable intervertebral disc prosthesis system in accordance with certain embodiments of the present invention having elbow-shaped extending portions shown in relation to an inner core;

FIG. 5B is an isometric view of the prosthesis system of 5A, showing an inner core partially disposed between superior and inferior endplate assemblies;

FIG. 5C is an isometric view of the prosthesis system of 5A in an applied position, showing an inner core fully disposed between superior and inferior endplate assemblies;

FIG. 6A is a side plan view of a slotted plate in accordance with certain embodiments of the present invention showing an elbow-shaped extending portion;

FIG. 6B is a side plan view of a slotted plate in accordance with certain embodiments of the present invention showing an L-shaped hook portion;

FIG. 6C is a side plan view of a slotted plate in accordance with certain embodiments of the present invention showing a circular extending portion;

FIG. 6D is a side plan view of a slotted plate in accordance with certain embodiments of the present invention showing a crooked extending portion;

FIG. 6E is a side plan view of a slotted plate in accordance with certain embodiments of the present invention showing an alternative placement for an extending portion;

FIG. 6F is an isometric view of an engagement plate in accordance with certain embodiments of the present invention showing an alternative configuration for an extending portion;

FIG. 7A is an isometric view of a superior endplate assembly in accordance with certain embodiments of the present invention in the extended position and having an arcuate profile;

FIG. 7B is an isometric view of a superior endplate assembly in accordance with certain embodiments of the present invention in the extended position and having a dome-like profile;

FIG. 7C is an isometric view of a superior endplate assembly in accordance with certain embodiments of the present invention in the extended position and having a circular shape;

FIG. 7D is a top view of a superior endplate assembly in accordance with certain embodiments of the present invention in the extended position and having a crescent shape;

FIG. 8 is a side plan view of a slotted plate in accordance with certain embodiments of the present invention having a spike protrusion for securing the slotted plate to the endplate of an adjacent vertebral body;

FIG. 9 is an isometric view of an endplate assembly in accordance with certain embodiments of the present invention in the extended position and having pores for promoting bone in-growth;

FIG. 10 is a side plan view through line 10-10 of FIG. 2A, showing a possible configuration for a slot;

FIG. 11 is a bottom view of a slotted plot in accordance with certain embodiments of the present invention showing a tapered slot configuration;

FIG. 12 is a bottom view of a slotted plate in accordance with certain embodiments of the present invention showing a slot having a two widths, a larger diameter entry point, and a larger diameter terminus;

FIG. 13 is a side plan view of a superior endplate assembly in accordance with certain embodiments of the present invention showing an engagement member extending through the engagement plate to the outer surface of the engagement plate;

FIG. 14A is a side plan view of a slotted plate in accordance with certain embodiments of the present invention showing a possible position of means for accepting an insertion tool;

FIG. 14B is a side plan view of a slotted plate in accordance with certain embodiments of the present invention showing another possible position of means for accepting an insertion tool;

FIG. 14C is a side plan view of a slotted plate in accordance with certain embodiments of the present invention showing still another possible position of means for accepting an insertion tool;

FIG. 15A is a front view of an inner core in accordance with certain embodiments of the present invention having curved projections;

FIG. 15B is a front view of an inner core in accordance with certain embodiments of the present invention having crooked projections;

FIG. 15C is a front view of an inner core in accordance with certain embodiments of the present invention having notched projections;

FIG. 15D is a front view of an inner core in accordance with certain embodiments of the present invention having a hourglass-like shape with a narrow stem;

FIG. 16A is a side view of a portion of an extendable intervertebral disc prosthesis in accordance with certain embodiments of the present invention including a set screw for preventing the inner core from shifting out of its applied position between endplate assemblies;

FIG. 16B is an isometric view of an extendable intervertebral disc prosthesis in accordance with certain embodiments of the present invention including a rigid member for preventing the inner core from shifting out of its applied position between endplate assemblies;

FIG. 17A is an isometric view of a superior endplate assembly of a telescoping intervertebral disc prosthesis system in accordance with certain embodiments of the present invention in the unextended position;

FIG. 17B is an isometric view of the superior endplate assembly of FIG. 16A in the extended position;

FIG. 17C is an isometric view of an outer member of a telescoping intervertebral disc prosthesis system in accordance with certain embodiments of the present invention;

FIG. 17D is an isometric view of an inner member of a telescoping intervertebral disc prosthesis system in accordance with certain embodiments of the present invention;

FIG. 17E is an isometric view of a telescoping intervertebral disc prosthesis system in accordance with certain embodiments of the present invention in an applied position showing an inner core between a superior endplate assembly and an inferior endplate assembly;

FIG. 18 is an isometric view of an insertion tool in accordance with certain embodiments of the present invention;

FIG. 19A is a top plan view of an insertion in accordance with certain embodiments of the present invention showing the tool in an undeployed configuration; and

FIG. 19B is a top plan view of an insertion tool in accordance with certain embodiments of the present invention in which a deployment block is used to engage the tool in a deployed configuration.

### DETAILED DESCRIPTION

The present invention generally relates to extendable intervertebral disc prosthesis systems, assemblies, and methods of replacing an intervertebral disc with an extendable intervertebral disc prosthesis. As used herein, the terms “inferior,” “superior,” “anterior,” “posterior,” “medial,” “lateral,” “upward,” “downward,” “top,” “bottom,” “horizontal,” “vertical,” “left,” and “right,” or vocabular modifications of such terms as well as other directional or anatomical orientation terms refer to positions and configurations of a disc replacement in an applied position when the patient is in the anatomical position (a term well known in the art). Further, an “applied position” refers to the position of the assembly when the slotted plate and the engagement plate have been inserted in the disc space of a patient. In embodiments where an inner core is included in the system and positioned between opposing endplates, the inner core holds the opposing endplates in place in an applied position of the system. By “extended position” is meant that a slotted plate of an endplate assembly is distracted in the lateral direction away from an engagement plate (or vice versa) of the assembly such that the assembly has moved from a position having a first width to a position having a second width greater than the first width. In the extended position, the slotted plate and the engagement plate have been fully distracted laterally away from one another (i.e., an engagement member has slid along the entire length of a slot and come to a rest at the medial-most point of the slot). By “unextended position” is meant that no engagement member has slid along the entire length of a slot. For example, an endplate assembly is in an unextended position when an engagement member rests against the lateral-most portion of the slot. However, any position that is not an “extended position” is an unextended position for purposes of this description.

Additionally, the terms “outer” and “inner” are used to described the opposing major surfaces on certain components of the present invention. As used herein, the term “outer” generally refers to a surface substantially parallel to the transverse plane of the body in an applied position and that abuts against the endplate of an adjacent vertebral body in an applied position of certain embodiments. As used herein, the term “inner” generally refers to a surface substantially parallel to the transverse plane of the body in an applied position and that opposes the outer surface. In an applied position of certain embodiments where an inner core is included, the inner surface abuts against an inner core. The orientation of the outer and inner surfaces will become more clear as the present invention is described in greater detail.

The present invention provides not only an extendable intervertebral disc prosthesis system but also various iterations of elements the endplate assemblies that make up the system. In describing this system, each of the assemblies will first be disclosed in detail. Additional elements of the assemblies will then be discussed, so that the initial elements of the system are described first, with additional elements being described thereafter.

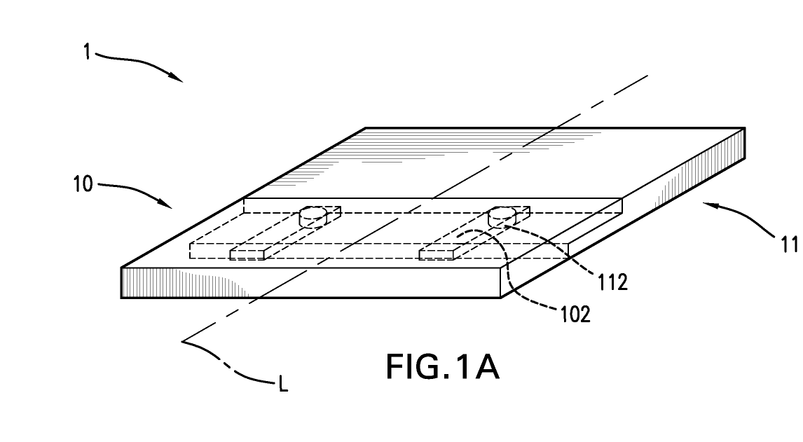

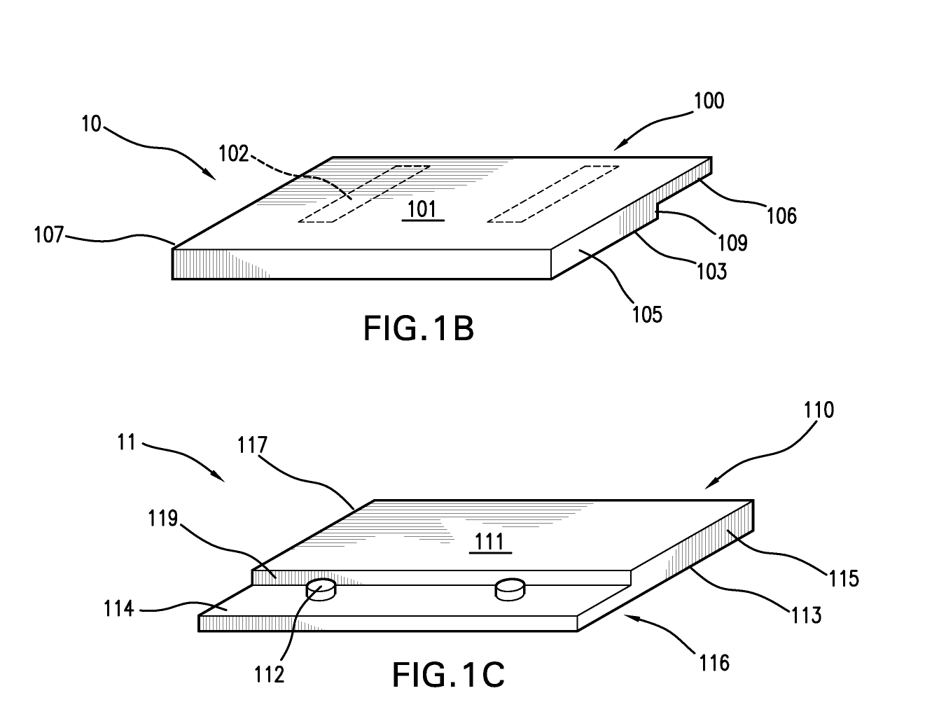

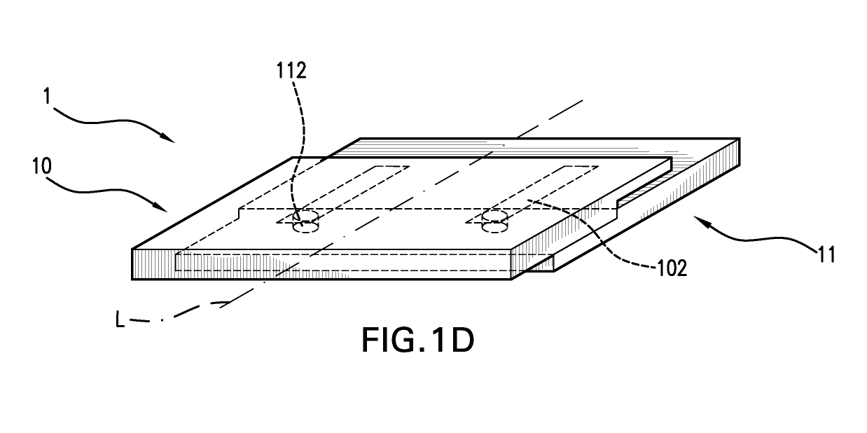

In certain embodiments, the present invention provides a multi-piece extendable intervertebral disc prosthesis endplate assembly that can be used individually. For example, referring to FIG. 1A, an endplate assembly 1 in accordance with certain embodiments of the present invention has a longitudinal axis L and comprises a slotted plate 10 and an engagement plate 11. As shown in FIG. 1B, a slotted plate of these embodiments includes a body 100 having an outer surface 101, an inner surface 103, a posterior surface 105, and an anterior surface 107. A slotted plate of these embodiments also has at least one slot 102, and preferably at least two slots, defined by at least inner surface 103. It should be noted that although at least one slot 102 in FIG. 1B is shown exposed to inner surface 103, the at least one slot could also be a slot extending through the slotted plate to the outer surface 101 (i.e. it could be a “through-slot”). In certain embodiments, the slotted plate may additionally comprise a lip 106 extending from a medial wall 109 of the slotted plate's body. As shown in FIG. 1C, an engagement plate 11 of these embodiments includes a body 110 having an outer surface 111, an inner surface 113, a posterior surface 115, an anterior surface 117, and a medial wall 119. Engagement plate 11 also includes a lip 116 extending from medial wall 119. Lip 116 has a contact surface 114. Engagement plate 11 further includes at least one engagement member 112 extending from contact surface 114. When slot 102 receives the at least one engagement member, the slotted plate slides relative to the engagement plate (or vice versa) along longitudinal axis L. As the plates slide apart from one another, the assembly moves from an unextended position (shown in FIG. 1D) to an extended position (shown in FIG. 1A).

The present invention also provides opposing endplate assemblies (hereinafter referred to as the “superior endplate assembly” and the “inferior endplate assembly”) for use individually, as part of an intervertebral disc prosthesis system (to be described in further detail below), or with other prostheses or assemblies known in the art. Each endplate assembly of these embodiments generally comprises the engagement plate and slotted plate described above and further comprises at least one extending portion. The extending portion may comprise an elbow, a hook-like shape, or other projections or protrusions in fluid communication with the plate's inner surface. In an extended position with an inner core disposed between opposing endplate assemblies, the extending portions act as a mechanism to prevent each endplate assembly from returning to its unextended position. Additionally, in an extended position with an inner core disposed between opposing endplate assemblies, the extending portions act as a mechanism to interlock each endplate assembly to an inner core (and thus, indirectly interlocks each endplate assembly to one another). Although not specifically mentioned for each embodiment, an extending portion may be located on the slotted plate, the engagement plate, or both the slotted plate and the engagement plate of each endplate assembly. Further, the extending portion on one endplate assembly need not have the same location, position, or configuration as the extending portion on the opposing endplate assembly. Numerous iterations and configurations are possible, and it should be understood that any combination falls within the scope of the present invention. In order to better understand the present invention, several preferred examples are provided below.

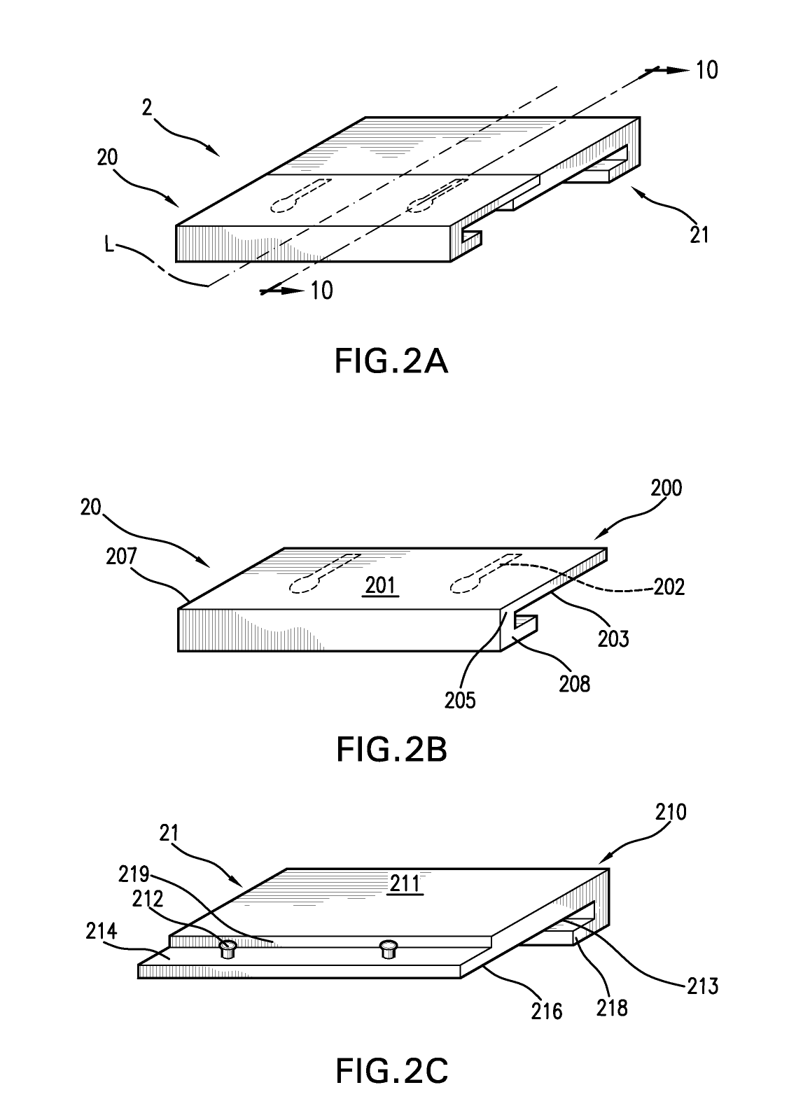

For example, FIGS. 2A-2C show an exemplary superior endplate assembly. This assembly 2 generally comprises a slotted plate (having a body 200 that includes an outer surface 201, an inner surface 203, a posterior surface 205, and an anterior surface 207) and engagement plate (having a body 210 that includes an outer surface 211, an inner surface 213, and a medial wall 219) as generally described above but also includes at least one extending portion. As shown in FIG. 2B, in addition to slot 202 a slotted plate 20 further comprises an extending portion 208 in fluid communication with inner surface 203 and projecting in an inferior direction when the superior endplate assembly is in an applied position. As shown in FIG. 2B, the extending portion is an L-shaped hook, but other shapes are also possible as discussed below. Also, in this embodiment, the extending portion is defined by a lateral surface of the slotted plate body, but this is not necessarily true for all embodiments. Referring to FIG. 2C, in certain embodiments, in addition to engagement member 212 an engagement plate 21 comprises an extending portion 218 in fluid communication with inner surface 213. As with the slotted plate, the extending portion is shown in FIG. 2C as an L-shaped hook and being defined by the lateral surface of the engagement plate body; however, different configurations are also possible. In certain preferred embodiments, both the engagement plate and the slotted plate comprise an extending portion as shown in FIG. 2A.

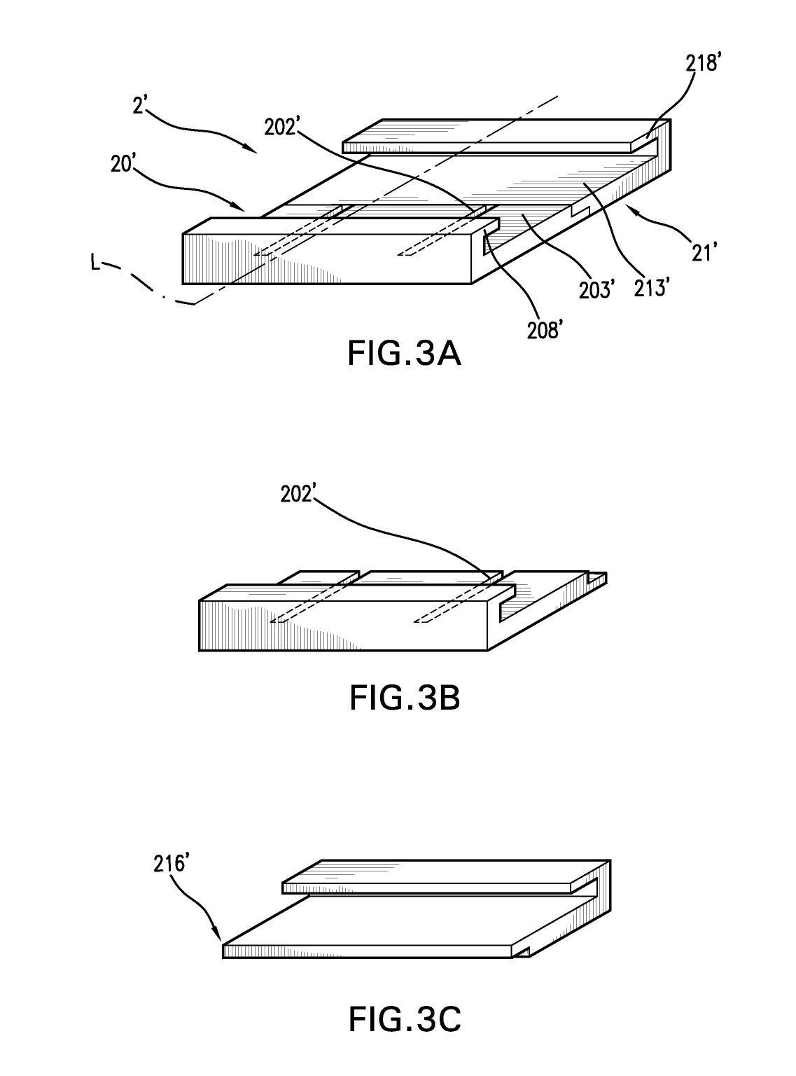

FIGS. 3A-3C show an inferior endplate assembly as well as components thereof. This assembly 2′ generally comprises a slotted plate and engagement plate as describe above but also includes at least one extending portion. As shown in FIG. 3B, in addition to slot 202′ a slotted plate 20′ further comprises an extending portion 208′ in fluid communication with inner surface 203′ and projecting in a superior direction when the inferior endplate assembly is in an applied position. As shown in FIGS. 3A-3C, the extending portion is an L-shaped hook, but other shapes are also possible as discussed below. Also, in this embodiment, the extending portion is defined by a lateral surface of the slotted plate body, but this is not true for all embodiments. Referring to FIG. 3C, in certain embodiments, in addition to engagement member 212′ an engagement plate 21′ further comprises an extending portion 216′ in fluid communication with inner surface 213′. As with the slotted plate, the extending portion is shown in FIG. 3C as an L-shaped hook and being defined by the lateral surface of the engagement plate body; however, different configurations are also possible. In certain preferred embodiments, both the engagement plate and the slotted plate comprise an extending portion as shown in FIG. 3A. Although an extending portion is shown on both the slotted plate and the engagement plate in FIG. 3A, this is not necessarily true for all embodiments, and the extending portion may be located on only one of the slotted plate and the engagement plate.

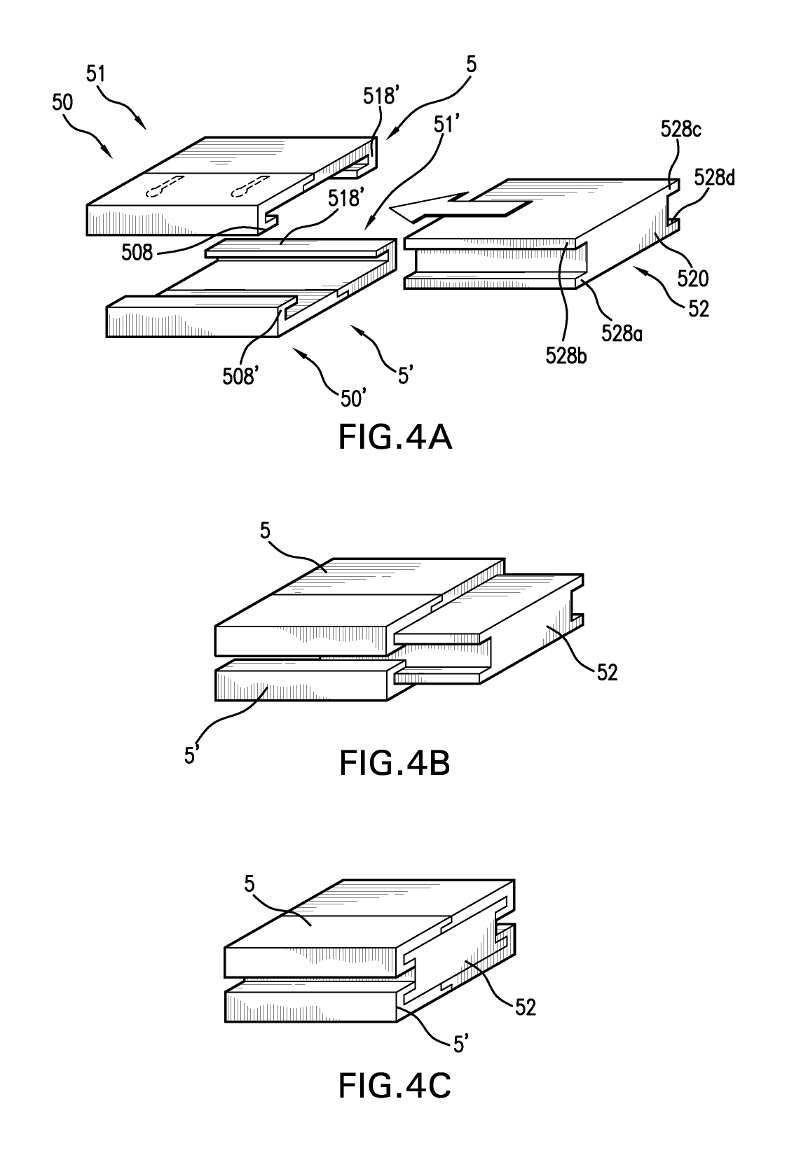

The present invention further provides an extendable intervertebral disc prosthesis system, comprising a superior endplate assembly, an inferior endplate assembly, and an inner core. In certain embodiments, the system generally comprises superior and inferior endplate assemblies as described above. An example of these embodiments is shown in FIG. 4A, wherein a prosthesis system includes a superior endplate assembly 5, an inferior endplate assembly 5′, and an inner core 52 configured to be disposed therebetween.

In certain applications, it may be necessary to hold the endplate assemblies in an extended position and also to interlock each of the endplate assemblies to the inner core. In embodiments of these applications, such as the prosthesis system shown in FIGS. 4A-4C, the slotted plates 50 and 50′ and the engagement plates 51 and 51′ of both the superior and inferior endplate assemblies 5 and 5′ comprise hook portions 508, 518, 508′, and 518′.

In these embodiments, an inner core 52 comprises a core body 520 and at least one projection extending from a surface of core body 520. Preferably, the at least one projection is a plurality of projections 528a-528d that allows the inner core to hold each of the superior and inferior endplate assemblies in the extended position and to interlock each endplate assembly to the inner core, thereby indirectly interlocking the superior endplate assembly to the inferior endplate assembly. In these embodiments, the inner core slides between the superior and inferior endplate assemblies much like a tongue slides into a groove, as shown in FIGS. 4A-4C. In preferred embodiments, the inner core is similar to the I-shaped configuration of inner core 52 shown in FIG. 4A where extending portions are on opposing lateral sides of the core body of both the superior and inferior endplate assemblies.

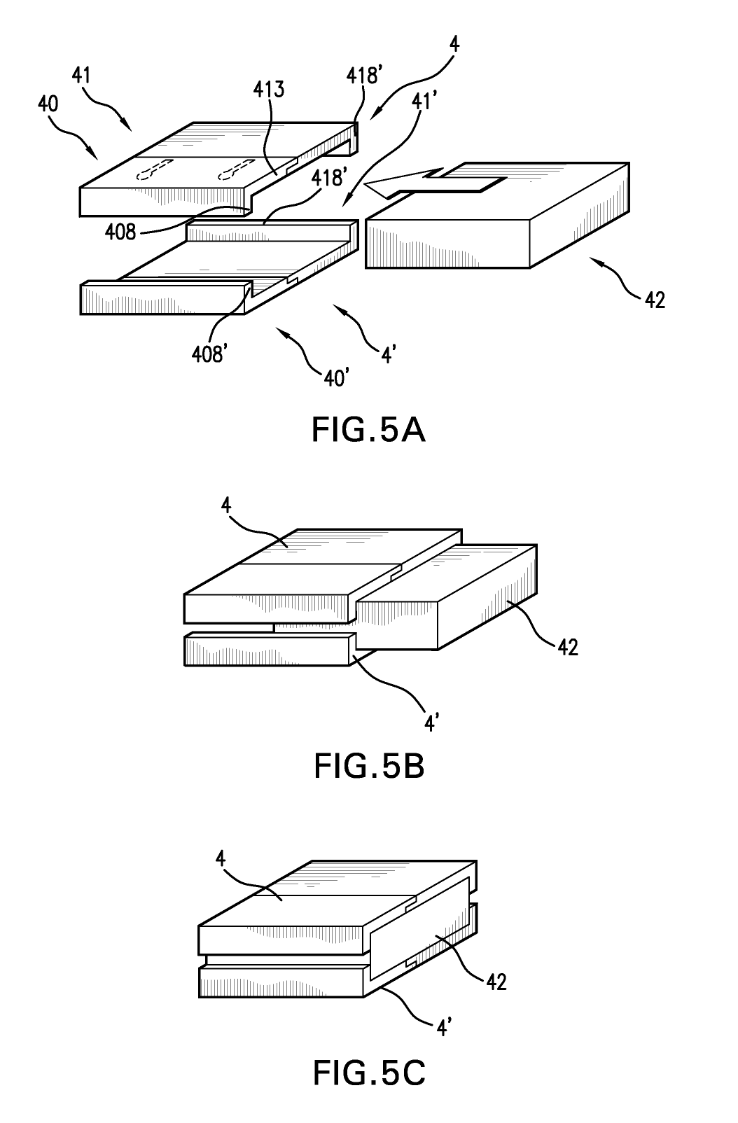

In certain other embodiments, such as the prosthesis system shown in FIGS. 5A-5C, slotted plates 40 and 40′ and engagement plates 41 and 41′ of both the superior and inferior endplate assemblies 4 and 4′ comprise extending portions 408, 418, 408′, and 418′. In these embodiments, the extending portions are elbows making a right angle with inner surface 403. In these embodiments, the elbows hold each of the endplate assemblies in an extended position, as the elbows of the endplate assemblies abut against the walls of the inner core (as shown in FIG. 5B and 5C), preventing the plates from moving toward one another and returning to an unextended position. In these embodiments, inner core 42 slides between endplate assemblies 4 and 4′, as shown in FIG. 5B. In the applied position of these embodiments, the inner core is completely disposed between endplate assemblies 4 and 4′, as shown in FIG. 5C although the posterior and anterior surfaces of the inner core need not be flush with the posterior and anterior surfaces of the endplate assemblies. Although the shape of the inner core of FIGS. 5A-5C is shown as a cube, this is not true for all embodiments, and the inner core may comprise any of a broad range of shapes that will allow it to abut to the extending portions of the endplate assemblies and hold the assemblies in an extended position.

Although the inner cores shown in FIGS. 4C and 5C have substantially the same depth as the endplate assemblies, this is not true for all embodiments. In certain embodiments, the inner core may have a depth greater than or less than the width of the endplate assemblies. Additionally, although the inner cores of these figures slide between their corresponding endplate assemblies in a posterior-anterior direction, other approaches are certainly possible, depending on the surgical approach used. For example, the core could be installed in an anterior to posterior direction or laterally.

As described above, in certain embodiments, the superior endplate assembly comprises extending portions in fluid communication with an inner surface of the slotted plate and/or the engagement plate and projecting in the inferior direction when the assembly is in an applied position, as shown in FIG. 2A. When an inferior endplate assembly comprises extending portions, the extending portions of an inferior endplate assembly project in the superior direction in an applied position, as shown in FIG. 3A.

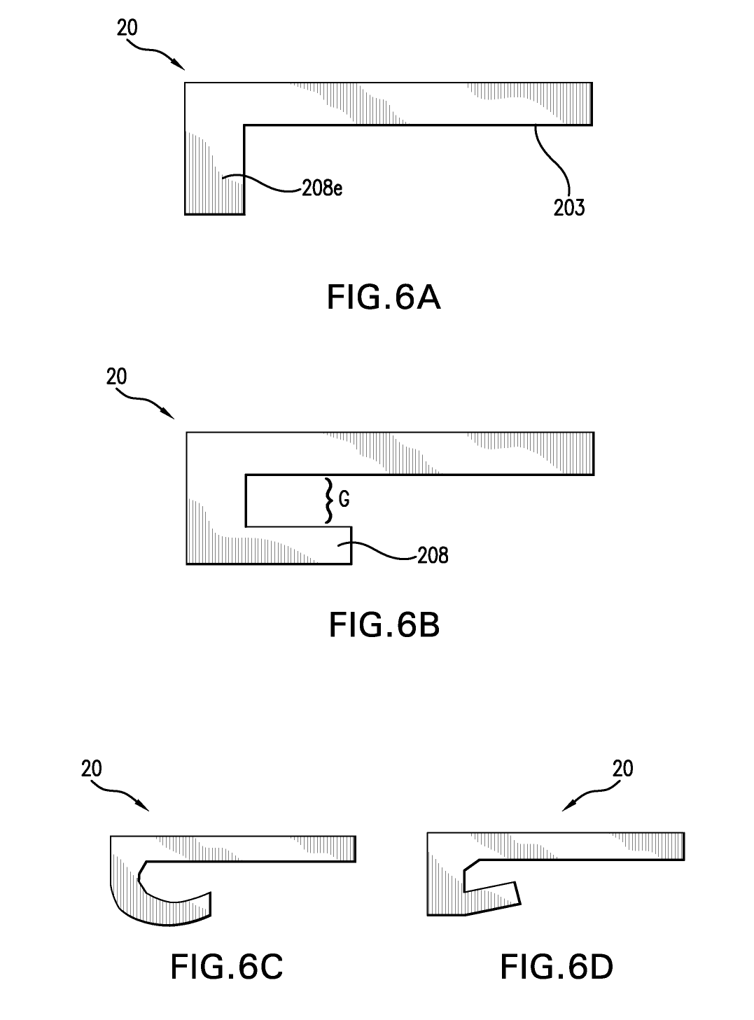

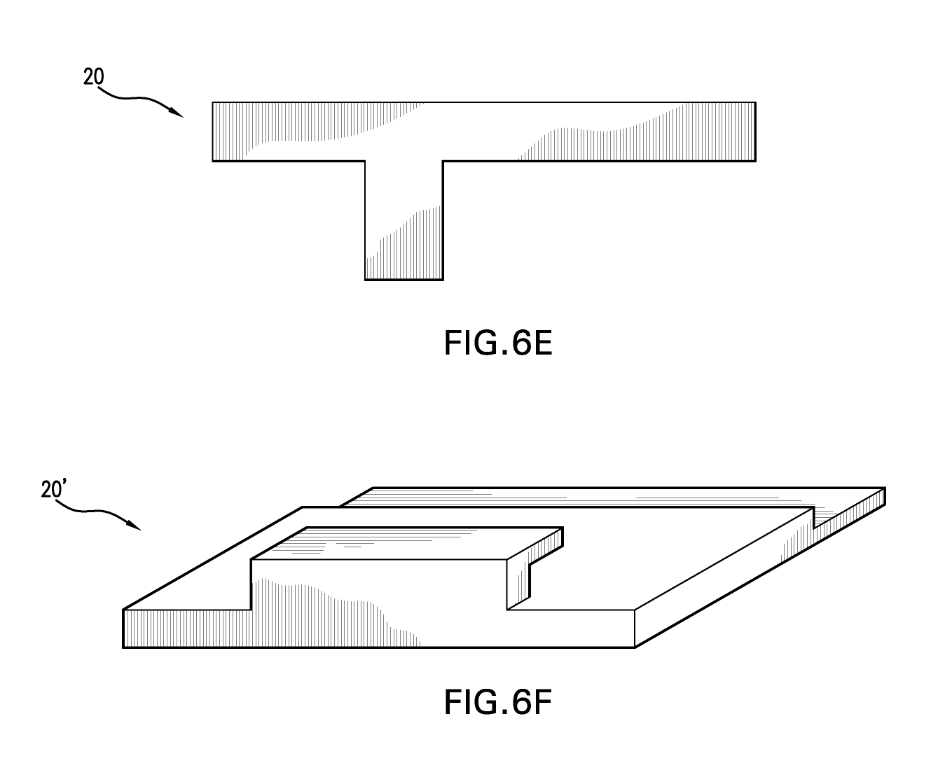

In certain embodiments, the extending portion is an elbow. Turning to FIG. 6A, an exemplary slotted plate 20 is shown with an elbow portion 208e as the extending portion. Although in FIG. 6A the elbow portion 208e is shown making a right angle with inner face 203, this need not be the case for all embodiments, and the elbow portion may make any angle with the inner face of the plate body. Preferably, the elbow portion makes a substantially right angle with an inner face that is substantially parallel to longitudinal axis L of an endplate assembly.

In certain other embodiments, the extending portion is a hook portion defining a groove extending along at least a portion of the space between the posterior and anterior surfaces of the body of the slotted plate, the engagement plate, or both. Turning to FIG. 6B, an exemplary slotted plate 20 is shown with a hook portion 208 defining a groove G. Although the hook portion in FIG. 6B is shown as having an L-shape projecting from the inner surface at a right angle, other shapes, configurations, and angles are possible. Non-limiting examples of alternative configurations include the curved configuration shown in FIG. 6C and the crooked configuration shown in FIG. 6D.

Although the hook and elbow portions have been described for a slotted plate, it should be understood that these descriptions hold true regardless of whether these components are located on the slotted plate or the engagement plate of an endplate assembly. Additionally, the configuration of these components need not be the same within each endplate assembly. Further, the extending portions (regardless of whether they are hooks, elbows, or some other configuration) need not be located towards the lateral-most surface of the plates and may instead be located closer to the middle of the plates, as shown in FIG. 6E. Also, the extending portions (regardless of whether they are hooks, elbows, or some other configuration) need not extend along the entire space between the anterior surface of a plate and the posterior surface of a plate but may instead extend through only a portion of this space, as shown in FIG. 6F.

Additionally, the extending portions need not be the same for the superior and inferior endplate assemblies or within the same endplate assembly (as shown in FIGS. 4A and 5A). For example, a superior endplate assembly could have a slotted plate with an elbow and an engagement plate with a hooked portion. In this example, the inferior endplate assembly could have a slotted plate with a curved hook portion and an engagement plate with an elbow.

The slotted plates and the engagement plates (including the lips and engagement members) of the present invention may be fabricated from any suitable biocompatible sterile material known in the art, including, but not limited to, metals, shape memory alloys, ceramic materials, polymeric materials, or any combination thereof. Non-limiting examples of suitable metallic materials include titanium, stainless steel, and cobalt chromium alloys. Non-limiting examples of suitable ceramic materials include zicronium oxide, aluminum oxide, and sintered silicon nitride. Non-limiting examples of suitable polymeric materials include polyarylesterketones, including polyetheretherketone (PEEK) and polyetherketoneketone (PEKK). The polymeric materials may also be reinforced with fillers or fibers, or may be oriented to provide additional mechanical properties. For example, the polymeric material can be reinforced with bioceramic or biolgass particles such as hydroxyaptite, which act as bioactive, bony in-growth agents and provide a reservoir of calcium and phosphate ions.

Slotted plates and engagement plates forming endplate assemblies are adapted to replace the removed intervertebral disc, and their respective bodies can have any suitable configuration that allows the endplate assemblies to fit within the intervertebral space at a given spinal level (such as at the sacral, lumber, thoracic or cervical level) and that allows the engagement plate to slide relative to the slotted plate along the assembly's longitudinal axis. In certain embodiments, the outer surfaces of the bodies of the slotted plates and the engagement plates match the shape and contour of the superior or inferior surfaces of adjacent vertebral endplates to better mate against the vertebral endplates. In certain embodiments, the bodies of the plates may have a planar outer surface that allows for more optimal or extended surface area contact with the adjacent porous or cancellous inferior or superior surface of an adjacent upper or lower vertebral body. In certain embodiments, the plates are configured such that the endplate assemblies in the extended position have a length of approximately 2.5-3.0 cm and a lateral width (along the longitudinal axis of the plates) of approximately 1.7-2.0 cm.

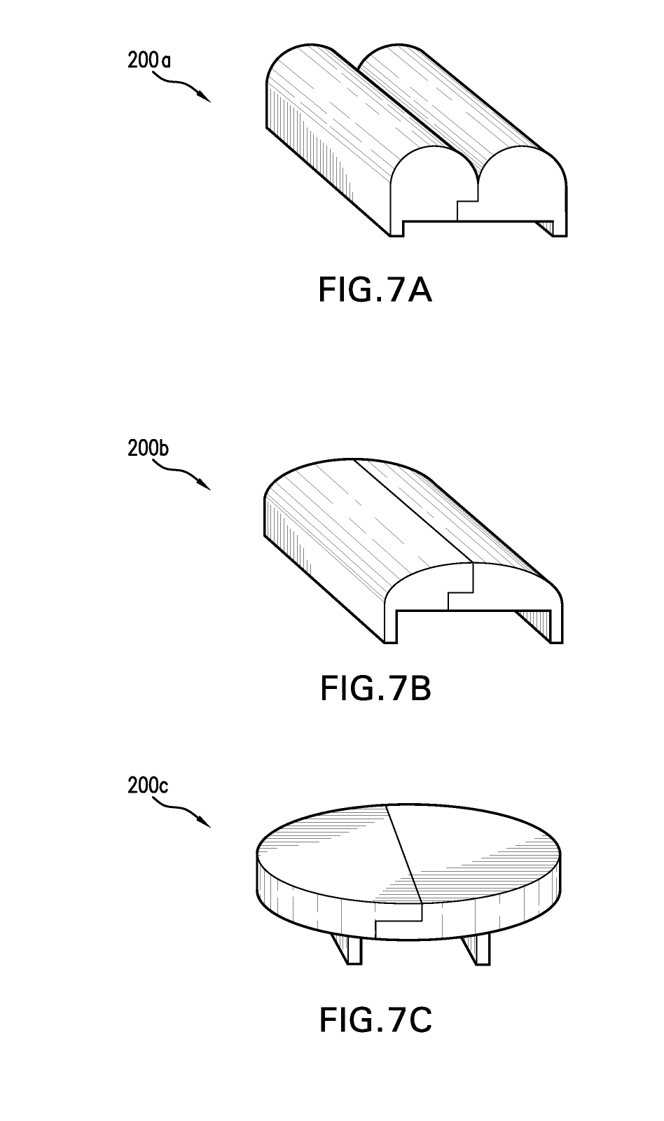

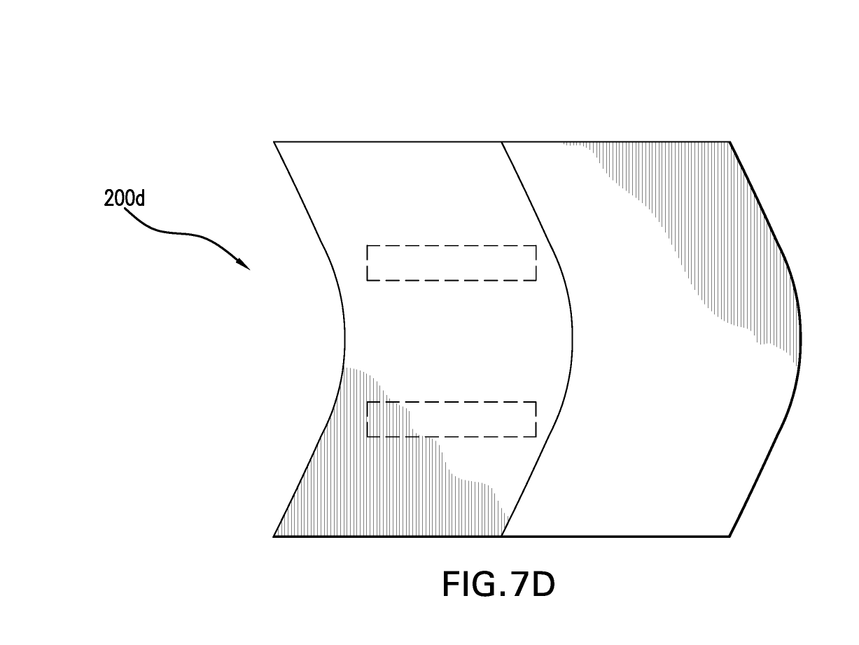

FIGS. 7A-7D illustrate several possible exemplary configurations for the outer surfaces of the bodies of the slotted plates and the bodies of the engagement plates. In each of FIGS. 7A-7D, a superior endplate assembly in the extended position is shown as a demonstrative example. It should be noted that any of the profiles depicted or described herein are equally applicable to both the superior and inferior endplate assemblies. It should also be noted that the placements and configurations of the extending portions shown in these figures are similarly demonstrative and do not represent the placement or configuration of this element in all embodiments of the present invention. The bodies of the plates of the present invention may have any of a broad variety of configurations, including, but not limited to, an arcuate profile (assembly 200a in FIG. 7A), a domed or convex-like profile (assembly 200b in FIG. 7B), a cylindrical profile (assembly 200c in FIG. 7C), a crescent-shaped profile (assembly 200d in FIG. 7D), or a rectangular profile (assembly 1 in FIG. 1A). The outer surfaces of the plates' bodies may also have a tapered thickness that increases in the anterior to posterior direction to provide an anterior to posterior lordotic taper to better restore the natural curvature of the spine. (Of course, depending on the orientation of the device, the taper can also be in the lateral direction to achieve the same lordotic taper.) These profiles are merely exemplary of the many possible configurations the plates may assume, and any other configuration suitable for use as an intervertebral disc replacement may be used. Any combination of suitable profiles may be used, and each endplate assembly need not have the same configuration. In addition, any of the endplate configurations described in U.S. Patent Application Publication No. 2008/0051902 filed on Aug. 8, 2007 (incorporated herein by reference in its entirety) may be suitable for use with the present invention.

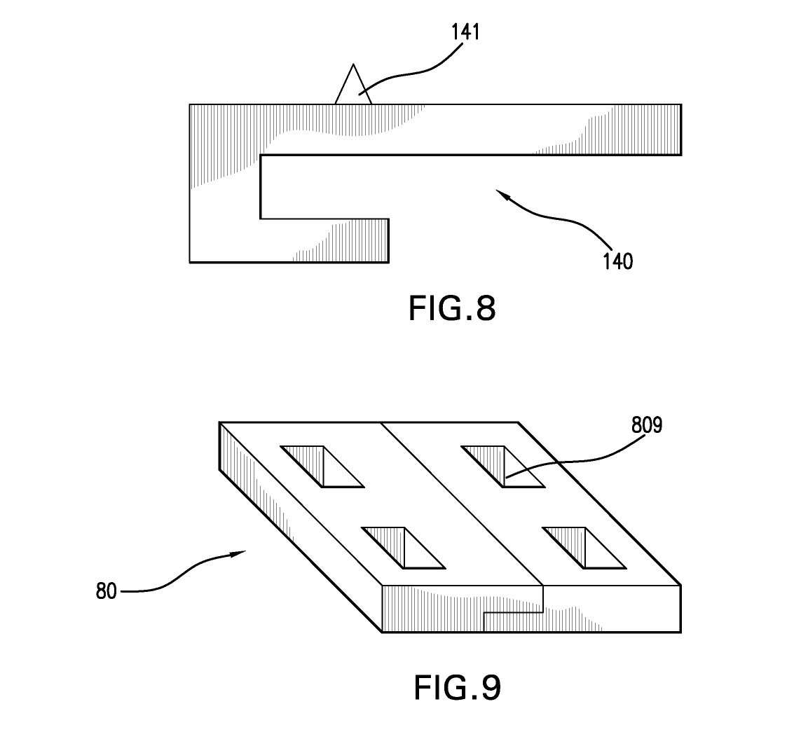

In certain embodiments, an endplate assembly of the present invention further comprises means for securing the prosthesis to adjacent superior or inferior vertebral bodies (hereinafter referred to as the “securing means”). In these embodiments, the securing means are located on the outer surface of the slotted plate, the engagement plate, or both the slotted plate and the engagement plate. The securing means are configured to anchor the endplate assembly in the intervertebral space and to prevent unwanted shifting of the prosthesis system after installation. Accordingly, the securing means may comprise any configuration that achieves this goal, including, but not limited to, a rough or jagged outer surface, the anchors described in U.S. Patent Application Publication No. 2008/0051902, a plurality of serrations, one or more spiked protrusions (such as protrusion 141 in FIG. 8), various biocompatible adhesives such as bone cement, and any suitable combination thereof.

In certain embodiments, the slotted plates and/or engagement plates comprise pores extending from the outer surface to the inner surface of the plates. A prosthesis system of these embodiments may be used to revise a disc prosthesis to an interbody fusion cage. Referring to FIG. 9, an exemplary endplate assembly 80 is shown having pores 809. The pores may comprise a variety of configurations and function much like pores 72 in FIG. 1 of U.S. Patent Application Publication No. 2008/0051902 cited above. The pores need not all assume the same configuration, and although rectangular pores are shown in FIG. 9, the pores may comprise any other configuration known to those skilled in the art as suitable for use with an interbody fusion device. These pores accommodate bone in-growth to provide solid fixation of the prostheses.

In embodiments where the endplate assemblies are fenestrated and thus are capable of being revised to an interbody fusion cage, the present invention provides for kits comprising endplate assemblies as generally described above and at least one, and preferably two, interbody fusion cages. The spinal fusion cage comprises a cage body and at least one projection extending from a surface of the cage body. The interbody cage can be inserted between the superior and inferior endplate assemblies, replacing the inner cores used in certain other embodiments. The interbody fusion cage can be any type known in the art such as, for example, a vertical fusion cage (such as a Harms cage) and a rectangular fusion cage (such as a Brantigan cage). Further descriptions of converting a disc replacement to an interbody fusion cage are described in U.S. Patent Application Publication No. 2008/0051902 cited above.

In certain embodiments, the outer surfaces of the bodies of the slotted plates and/or the engagement plates include a porous coating or osteoconductive mesh structure. Alternatively, the surfaces can be made porous, such as by titanium plasma spray. For example, the outer surfaces may comprise a titanium bead coating applied via spraying or sintering. Alternatively, outer surfaces of the plates' bodies can be roughened in order to promote bone in-growth into the defined roughened surfaces of the disc prosthesis.

The porous layer or surface on the outer surfaces of the plates' bodies may also deliver desired pharmacological agents. The pharmacological agent may be, for example, a growth factor to assist in the repair of the vertebral endplates and/or the annulus fibrosis. Non-limiting examples of growth factors include a bone morphogenetic protein, transforming growth factor (TGF-β), insulin-like growth factor, platelet-derived growth factor, fibrolast growth factor, or other similar growth factor or combinations thereof having the ability to repair the endplates an/or the annulus fibrosis of an intervertebral disc.

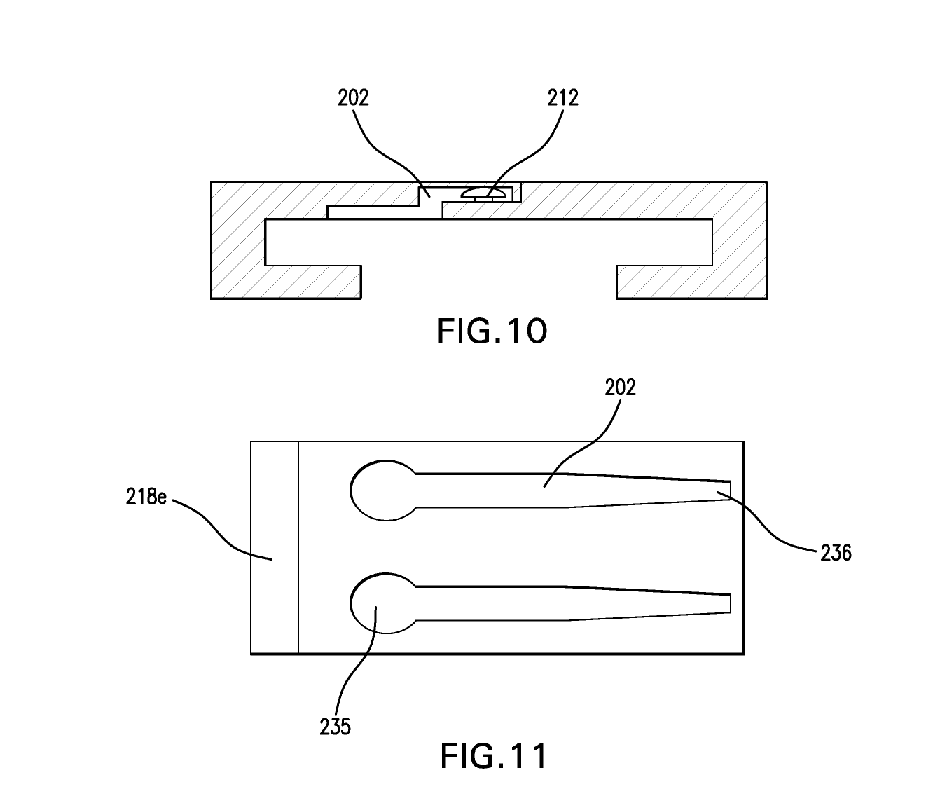

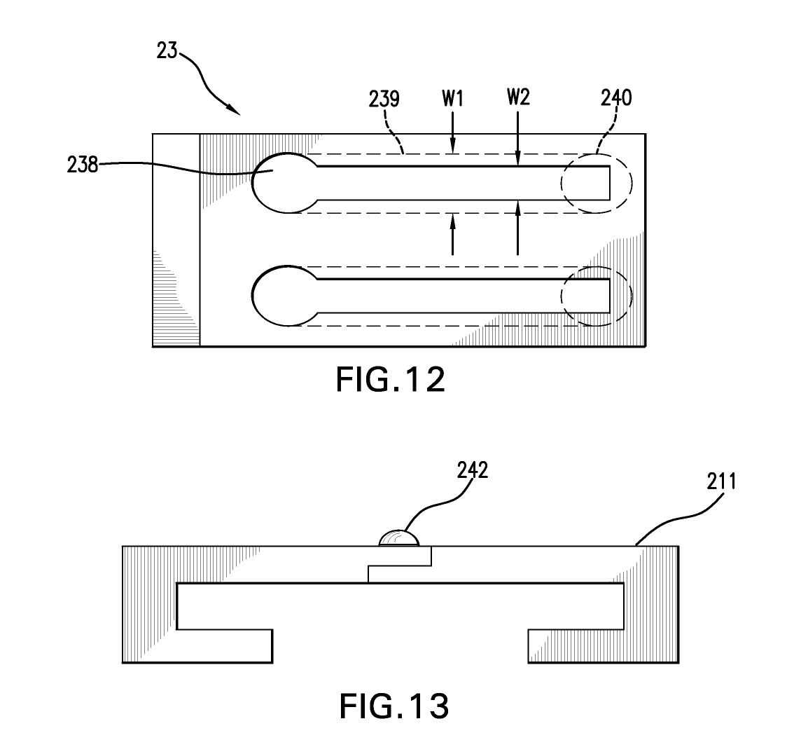

Regarding further details of the slot(s) of a slotted plate of an endplate assembly according to certain embodiments of the present invention, as described above, the at least one slot of an endplate assembly extends longitudinally across the slotted plate. The slot extends from the inner surface of the slotted plate toward the outer surface of the slotted plate, although it need not comprise a through hole in all embodiments. In certain embodiments, the depth of the slot may not be constant throughout, as shown in FIG. 10. FIG. 10 shows a cut away view through slot 202 of FIG. 2B. The slot should have a width that can accommodate the engagement member of the engagement plate, although the slot may comprise an entry portion 235 having a wider width at its lateral-most end (farthest from the engagement plate), such that the slot resembles the key-hole shape shown in FIG. 11. Additionally, the at least one slot may comprise a wider diameter at its lateral-most end, as shown by terminus 240 in FIG. 12. The larger diameters at locations 235 in FIG. 11 and 238 and 240 in FIG. 12 may be configured, for example, to accommodate an engagement member having a larger diameter at its top portion, such as engagement member 212 in FIG. 2C. In preferred embodiments, the at least one slot comprises two slots located on the inner surface of the slotted plate's body and configured and positioned to receive two corresponding engagement members located on the engagement plate. However, the at least one slot may also comprise one slot or three or more slots. Also, in preferred embodiments, the at least one slot does not extend to the medial-most end of the slotted plate, thereby preventing the engagement plate from separating from the slotted plate when the plates are distracted laterally.

In certain embodiments, the at least one slot has a tapered width such that the width narrows in the longitudinal direction. In these embodiments, the narrowest point of the slot 236 is at the medial-most end of the slotted plate (the end closest to the engagement plate), as shown in FIG. 11. As the engagement members slide down the slots of these embodiments, the width of the slot narrows. When an engagement member reaches point 236, the width of the slot is slightly less than the width of the engagement member, resulting in an interference fit that holds the endplate assembly in the extended position. Other geometric configurations apparent to one of ordinary skill in the art may also be used to hold the endplate assembly in the extended position, and the tapered geometry described above provides merely one example.

In certain embodiments, the at least one slot of the slotted plate extends through the inner surface of the slotted plate to the outer surface of the slotted plate at the medial-most end of the slot. In these embodiments, the at least one engagement member 242 extends above outer surface 211 of the engagement plate (as shown in FIG. 13) in an extended position. The at least one engagement member of these embodiments may further comprise one or more of the securing means described above, allowing the at least one engagement member to anchor the endplate assembly to an adjacent vertebral body. For example, in certain embodiments the at least one engagement member may comprise a spike, similar to protrusion 141 in FIG. 8.

Regarding a lip of an engagement plate of an endplate assembly according to certain embodiments of the present invention, such a lip serves as a platform on which the at least one engagement member rests. As shown in FIG. 2C, lip 216 has a contact surface 214 from which engagement member 212 projects. Although the lip is shown as extending in a substantially perpendicular direction from the medial wall this need not be the case for all embodiments, and in certain embodiments the lip may project at an angle relative to the inner surface of the engagement plate. The lip may be fabricated from the same material as the body of the engagement plate, or it may be fabricated from any other suitable sterile biocompatible material.

The at least one engagement member of an engagement plate is configured to be received by the at least one slot of the slotted plates and projects away from the contact surface of a lip of an engagement plate. The at least one engagement member may comprise a broad variety of configurations, and non-limiting examples include rectangular, cylindrical, mammilated, hourglass-like, or mushroom-like shapes. The engagement members can have the same cross-sectional profile throughout their length, but in other embodiments the cross-sectional profile may vary throughout the length similar to engagement member 212 in FIG. 2C. Although the engagement member in FIG. 2C is shown projecting in a substantially perpendicular direction from the contact surface of the lip this is not true for all embodiments, and in other embodiments not shown here the at least one engagement member may project at an angle relative to the contact surface. The engagement members may be fabricated from any suitable sterile biocompatible material. They may, for example, be manufactured via a molding procedure, such as injection molding, during manufacture of the engagement plate or may be a separate component attached to the lip of the engagement plate by various known appropriate means such as welding, an interference fit, or various threaded fastening systems.

Preferably, the at least one engagement member of the engagement plate is a mushroom-like shape, similar to engagement member 212 of FIG. 2C. In these embodiments, an example of which is shown in FIG. 12, a slot 239 of a slotted plate 23 would have a wider diameter at its lateral-most end 238 and a wider diameter at it's medial-most end 240 configured to accommodate the wider head of an engagement member. In these embodiments, slot 239 may comprise a first width W₁ configured to accommodate the wider head portion of an engagement member and a second, thinner width W₂ configured to receive the thinner stem of an engagement member. Width W₂ is thinner than the width of the head of the engagement member, such that the engagement member can only enter or exit slot 239 at end 238.

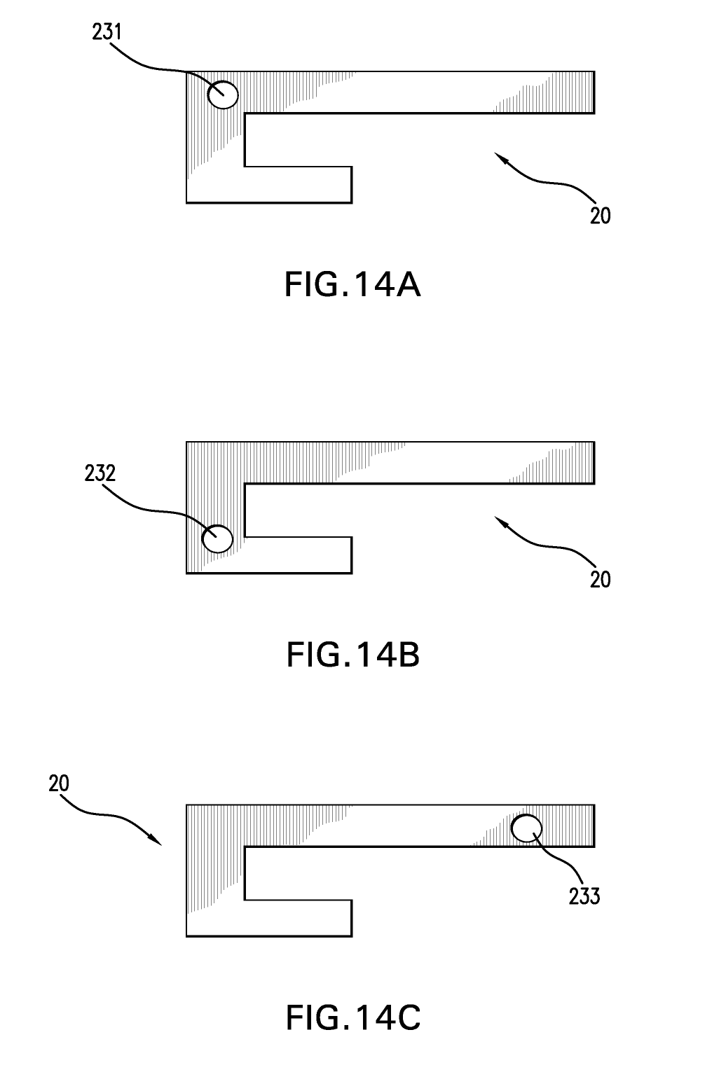

In certain embodiments, the endplate assemblies may further comprise means for accepting an insertion tool (hereinafter referred to as the “accepting means”). In certain embodiments, the insertion tool is used not only to impact the endplate assemblies into the intervertebral disc space but also to distract each assembly into the extended position. Preferably, the accepting means are located on the anterior or posterior surfaces of the slotted plates and/or the engagement plates. However, the accepting means may be located on other surfaces of the slotted plates or engagement plates in other embodiments. The accepting means may be any means known in the art suitable for receiving the distal end of a surgical insertion tool in order to impact the endplate assemblies into the appropriate position in the intervertebral disc space. Possible accepting means include (but are not limited to) one or more holes, grooves, channels, slots, any other type of recess of appropriate configuration for removable attachment to the distal end of an insertion tool, or any suitable combinations thereof. The accepting means may also be one or more protrusions appropriately configured for removable attachment to the distal end of an insertion tool, including (but not limited to) pegs of various shapes and sizes. The protrusions or recesses may be threaded, as appropriate, or may include the use of magnets to secure the endplate assemblies to an insertion tool. Turning to FIG. 14A, in certain embodiments, the accepting means are recesses 231 located on the posterior surfaces of slotted plate 20 or an engagement plate. In other embodiments (not shown), the accepting means may be located on the anterior surfaces of the slotted plates and the engagement plates. Alternatively, the accepting means may be located on a posterior or anterior surface of the extending portion, as shown by recess 232 in FIG. 14B. Preferably, the accepting means is located on an anterior or posterior surface of the plate toward the medial end of plate 20, as shown by recess 233 in FIG. 14C. The accepting means may be a combination of any of the configurations mentioned above or any other means known in the art suitable for accepting the distal end of a surgical insertion tool in order to impact the endplate assembly into the appropriate position in the intervertebral disc space and distract the assembly into the extended position.

Although the slotted plates are shown on the left and the engagement plates are shown on the right in the examples shown in the figures, the plates' positioning could of course be reversed.

Regarding further details of an inner core of an endplate system of the present invention, the inner core of an endplate assembly may comprise any suitable biocompatible sterile material including, but not limited to, various polymers and plastics (such as polyethylene), metals, alloys, and ceramic materials. In addition, the inner core may comprise a composite material or other combinations of materials such that certain material properties (e.g., the modulus of elasticity) are not constant or homogeneous throughout the entire core. Preferably, the inner core comprises a somewhat flexible material or combination of flexible materials, such as an elastomeric material, allowing it to mimic the functionality of the nucleus pulposus of a natural intervertebral disc. The body of the inner core may comprise any configuration that allows it to be at least partially contained between the endplate assemblies and, in appropriate embodiments, hold the endplate assemblies in the extended position and interlock each endplate assembly to the inner core.

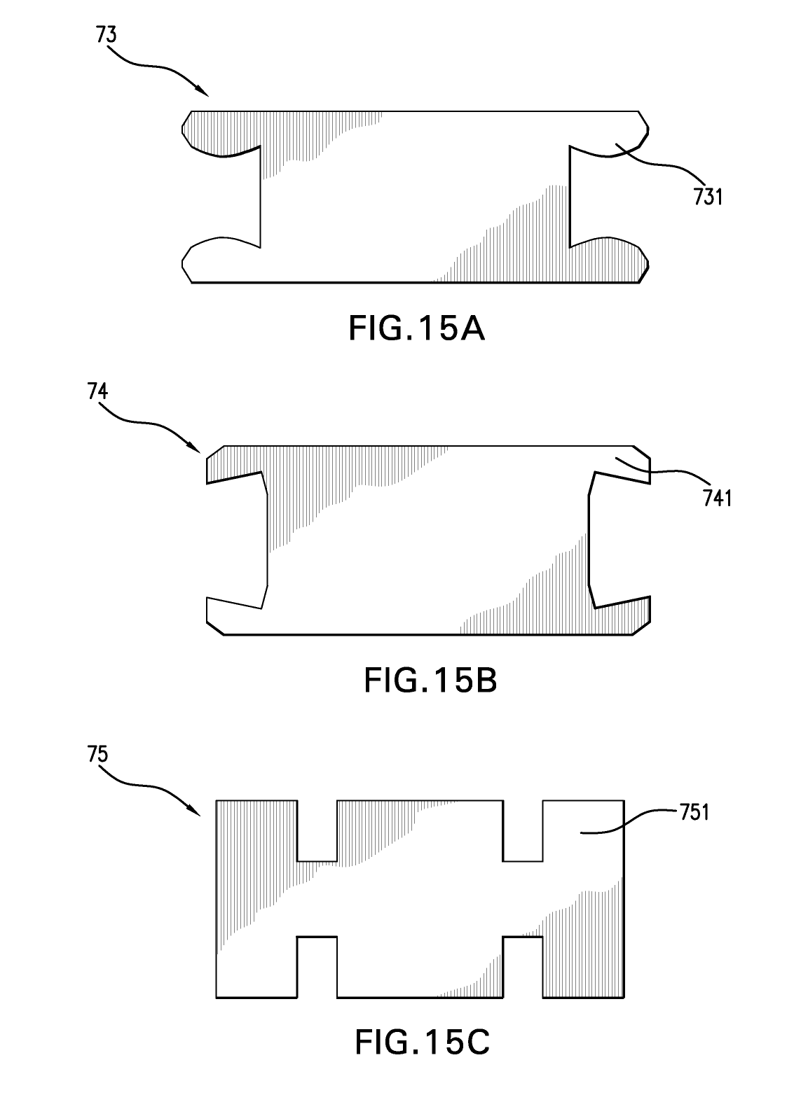

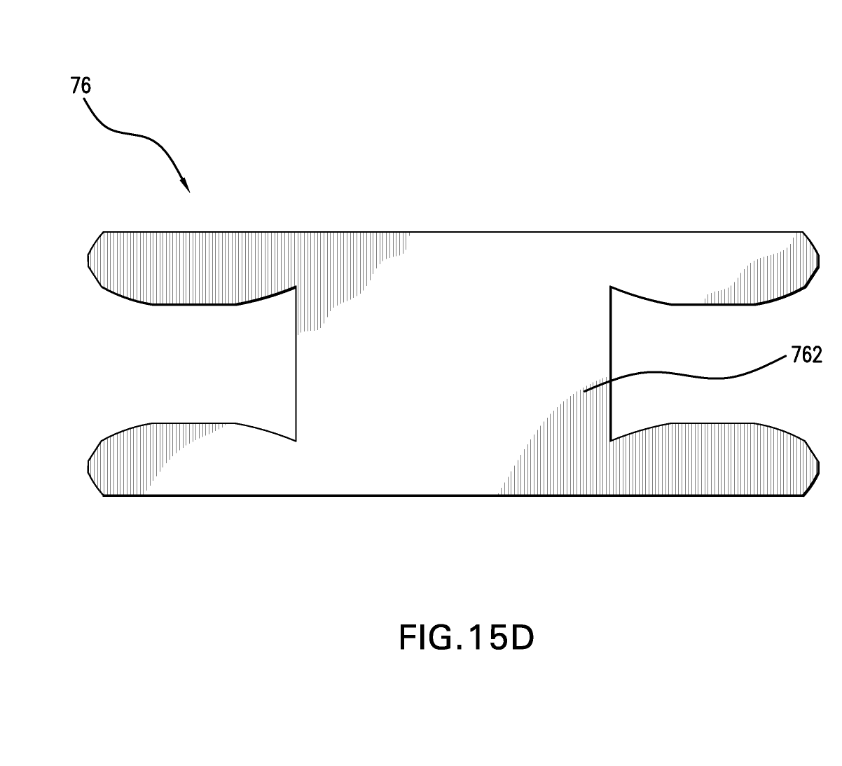

In certain embodiments, the inner core comprises at least one projection. In certain embodiments, the at least one projection is four rectangular projections, such as projections 528 shown in FIG. 4A. However, other configurations for the at least one projection are also possible. For example, the at least one projection of certain embodiments resembles the circular shape of projection 731 of inner core 73 shown in FIG. 15A, the crooked or tapered shape of projection 741 of inner core 74 shown in FIG. 15B, or the notched configuration of projection 751 of inner core 75 shown in FIG. 15C. These shapes would be received by, for example, the corresponding extending portions shown in FIGS. 6C-6E described above. Additionally, in certain embodiments the inner core may comprise an hourglass-like shape such as that of inner core 76 shown in FIG. 15D. In these embodiments, the inner core may have a narrow middle portion, such as middle portion 762.

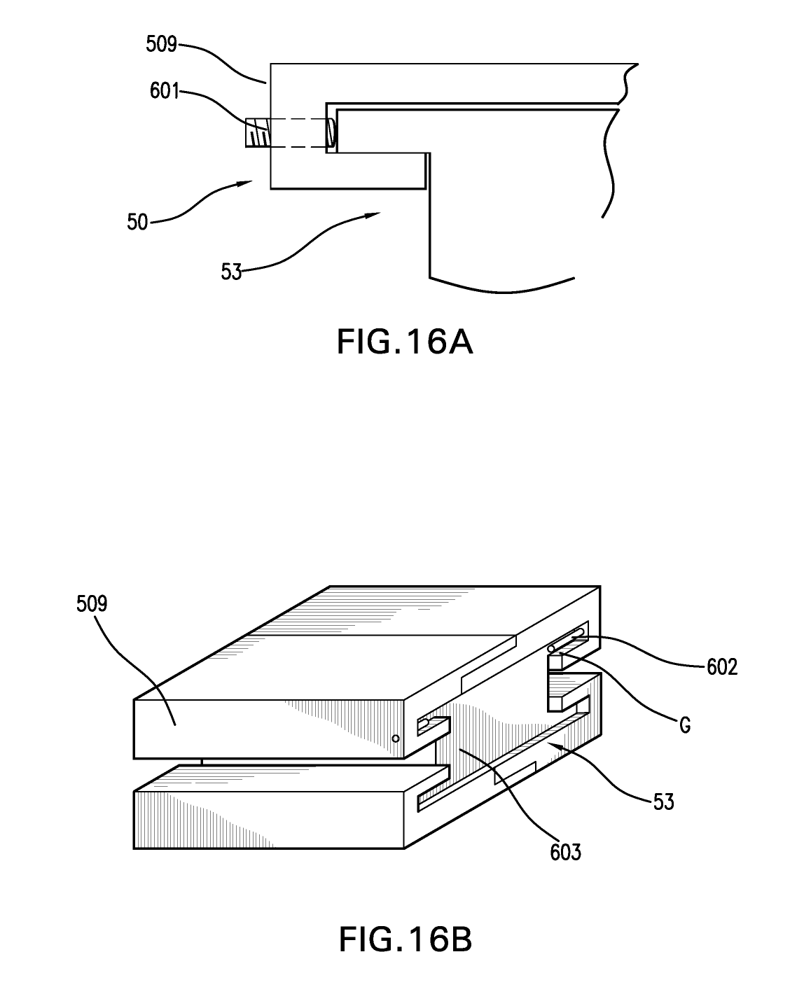

An extendable intervertebral disc prosthesis in accordance with certain embodiments of the present invention may further comprise means for preventing the inner core from shifting out from the proper position between the superior and inferior endplate assemblies (hereinafter referred to as the “preventing means”). By “proper position” it is meant the position of the inner core between the superior and inferior endplate assemblies such that the inner core holds each endplate assembly in the extended position. In addition, in the “proper position” no part of the inner core protrudes beyond the plane created by the anterior surfaces of the superior and inferior endplate assemblies and the plane created by the posterior surfaces of the superior and inferior endplate assemblies. In certain embodiments, the preventing means may comprise at least one set screw located on one or more surfaces of the slotted plates, the engagement plates, or both. For example, as shown in FIG. 16A, in certain embodiments a set screw 601 extends through a hole in lateral surface 509 of plate 50 to abut to a lateral surface of inner core 53. In other embodiments, as shown in FIG. 16B, the anterior to posterior length of the inner core may be less than the anterior to posterior length of the endplate assemblies, allowing a set screw or other rigid member to extend into a portion of groove G. In these embodiments, a major surface of rigid member 602 abuts to the posterior or anterior surface (surface 603 in FIG. 16B) of inner core 53. The rigid member of these embodiments may comprise a dowel, pin, or similar rigid member that extends through or from one or more of the lateral surfaces of the endplate assemblies, as shown by rigid member 602 in FIG. 16B. Additionally, the preventing means may comprise a roughened portion located on a superior or inferior surface of an inner core or on an outer or inner surface of an engagement or slotted plate. In these embodiments, the roughed surface or surfaces are configured to frictionally engage one another such that the inner core is prevented from shifting out of the proper position between the endplate assemblies. Although several examples for the preventing means have been provided here, these examples are in no way limiting, and the preventing means may comprise any suitable configuration, structure, or additional component that prevents the inner core from shifting out from the proper position between the endplate assemblies.

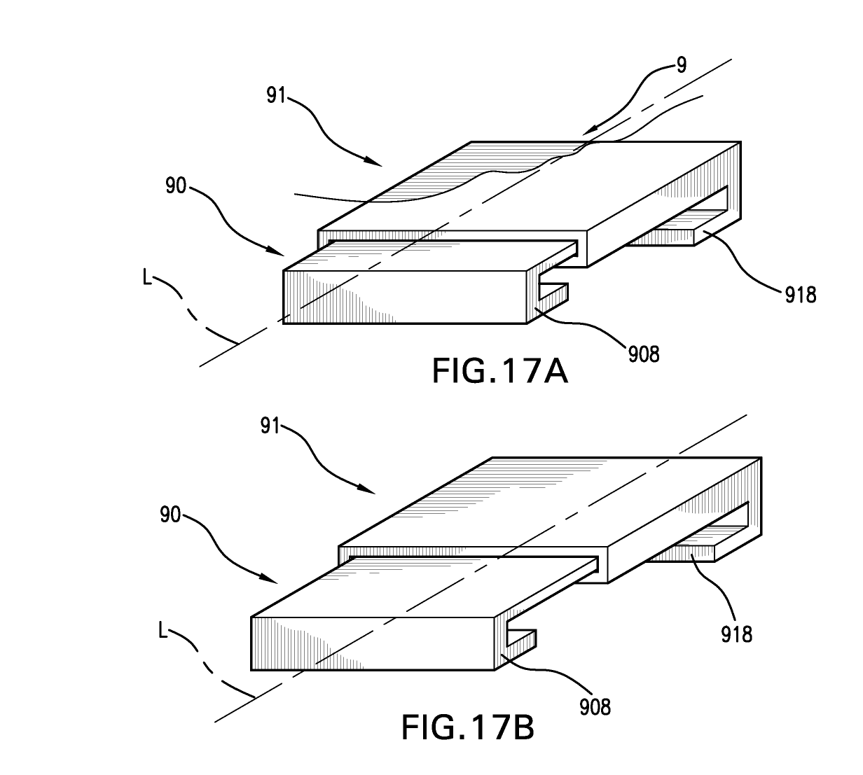

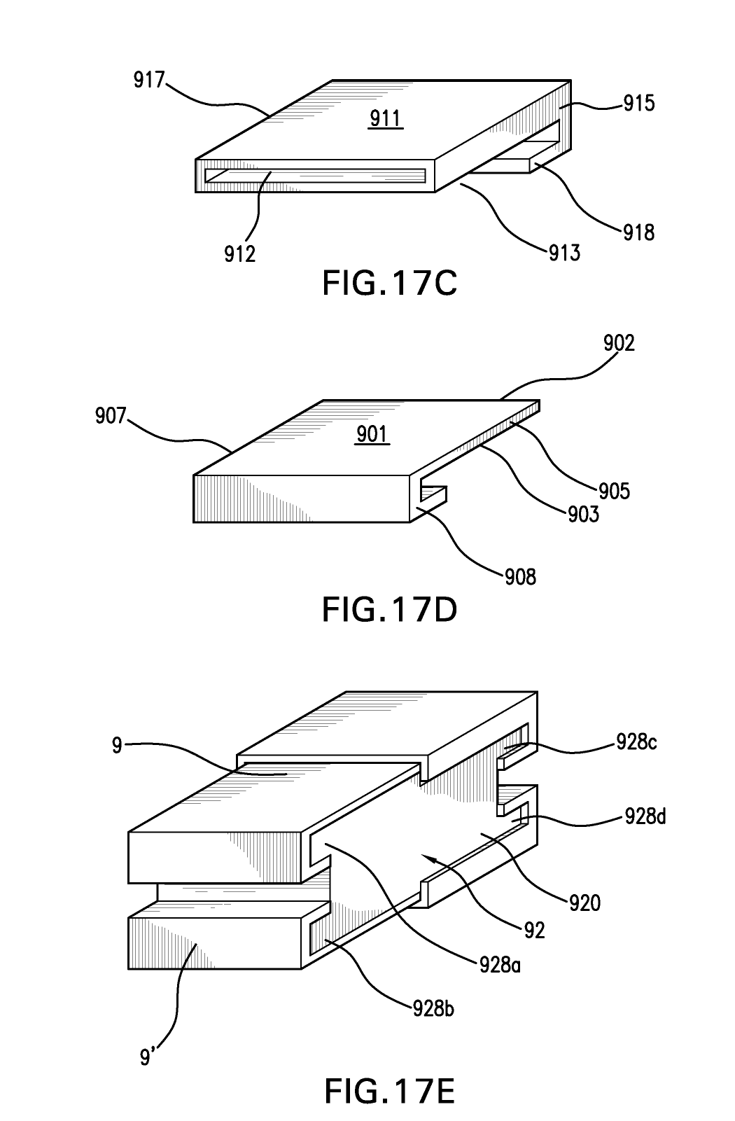

The present invention further provides a telescoping intervertebral disc prosthesis system. Turning to FIGS. 17A-17E, in certain embodiments, a telescoping intervertebral disc prosthesis system comprises a superior endplate assembly 9, an inferior endplate assembly 9′, and an inner core 92. Each endplate assembly of these embodiments has a longitudinal axis L and comprises an inner member 90 and an outer member 91. Inner member 90 includes a body (having a cross-sectional profile 902, an outer surface 901, an inner surface 903, a posterior surface 905, and an anterior surface 907) and a hook portion 908 defining a groove extending along at least a portion of the space between posterior 905 and anterior surface 907. Outer member 91 includes a body (having an outer surface 911, an inner surface 913, a posterior surface 915, and an anterior surface 917), a longitudinal channel 912 defined by a space large enough to receive cross-sectional profile 902, and a hook portion 918 defining a groove extending along at least a portion of the space between posterior surface 915 and anterior surface 917. When inner member 90 slides within longitudinal channel 912, endplate assembly 9 moves from an unextended position (shown in FIG. 17A) having a first width to an extended position (shown in FIG. 17B) having a second width greater than the first width.

In these embodiments, inner core 92 includes a core body 920 and projections 928a-928d (collectively referred to as 928) extending from a surface of core body 920. The projections 928 are configured to be slidably received by the grooves of hook portions 908 and 918 (as well as the hook portions on assembly 9′). When the inner core slides between the superior and inferior endplate assemblies, the endplate assemblies are each held in the extended position and are each interlocked to the inner core.

Any of the additional elements described above for an extendable intervertebral disc prosthesis system (such as, for example, the pores, the securing means, the preventing means, and the accepting means) may be added to these embodiments. The inner and outer members of these embodiments may be fabricated from any of the sterile, biocompatible materials suitable for the slotted plates and the engagement plates above. Similarly, the inner cores of these embodiments may be fabricated from any of the sterile, biocompatible materials suitable for the inner cores of the previously mentioned embodiments. Additionally, the inner cores of these embodiments may be replaced with an interbody fusion cage for conversion to a fusion device in much the same manner mentioned above. Although the inner member is shown on the left and the outer member is shown on the right in the illustrated examples, the members' positioning could of course be reversed.

The present invention also provides methods for surgically implanting an extendable intervertebral disc prosthesis in an intervertebral disc space. In certain embodiments, a method comprises the steps of removing a patient's natural intervertebral disc from the intervertebral disc space. The method further comprises providing a superior endplate assembly and an inferior endplate assembly each capable of assuming an unextended position and an extended position. The method then comprises implanting in the unextended position either one of the superior endplate assembly or the inferior endplate assembly in the excised disc space and distracting the either one of the superior endplate assembly or the inferior endplate assembly to an extended position. The method further comprises implanting in the unextended position the other of the superior endplate assembly or the inferior endplate assembly in the excised disc space and distracting the other of the superior endplate assembly or the inferior endplate assembly to an extended position. The method additionally comprises implanting an inner core between the superior and inferior endplate assemblies.

In certain embodiments, an insertion tool capable of simultaneously inserting and simultaneously distracting the endplate assemblies is used to implant the endplate assemblies. This insertion tool may comprise four insertion fingers capable of engaging with the insertion means of each of the slotted plates and the engagement plates. In certain embodiments, the insertion tool may resemble a four-fingered pair of tongs.

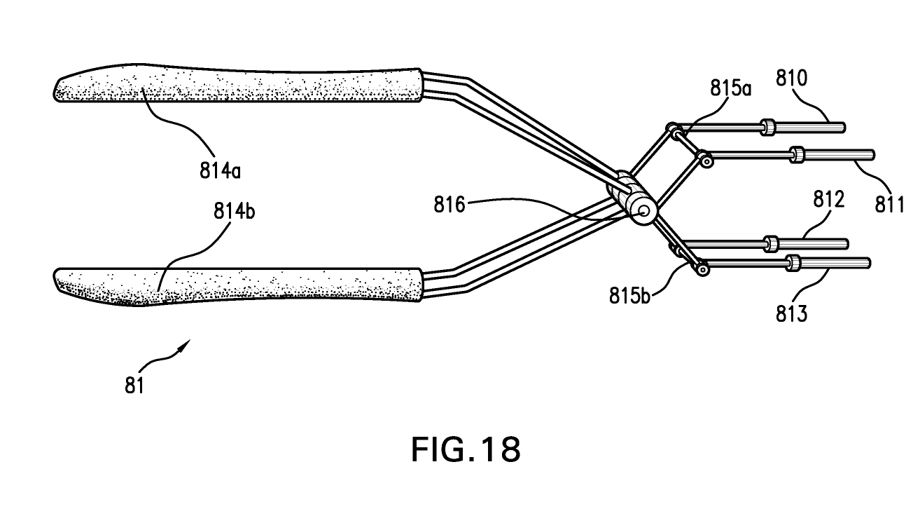

The present invention also provides a kit for replacing a natural intervertebral dic with an extendable intervertebral disc prosthesis system. A kit of these embodiments comprises an extendable intervertebral disc prosthesis system (such as those described above) and an insertion tool. In certain embodiments, such as the embodiment shown in FIG. 18, insertion tool 81 includes a plurality of insertion fingers (including left superior insertion finger 810, right superior insertion finger 811, left inferior insertion finger 812, and right inferior insertion finger 813), means for laterally distracting the left insertion fingers from the right insertion fingers (hereinafter “distracting means”), and a pair of handles 814a and 814b pivotally connected (at point 816) to the distracting means. The handles are capable of distracting the superior insertion fingers from the inferior insertion fingers in the superior-inferior direction, thus allowing the user to obtain a proper height for the endplate assemblies of an extendable intervertebral disc prosthesis system prior to the insertion of an inner core. The fingers are configured to attach to superior and inferior endplate assemblies of an extendable intervertebral disc prosthesis system. In certain embodiments, the fingers comprise means for attaching the fingers to superior and inferior endplate assemblies. These means may comprise a variety of configurations, including (but not limited to) threaded engagements, male-female connection systems, any of the configurations suitable for the insertion means described for the extendable intervertebral disc prosthesis system above, or any other means suitable for attaching the fingers to the plates of an extendable intervertebral disc prosthesis system. In certain embodiments, the means for attaching are configured to interact with the insertion means described above. The plurality of fingers is connected (either directly or indirectly) to the distracting means.

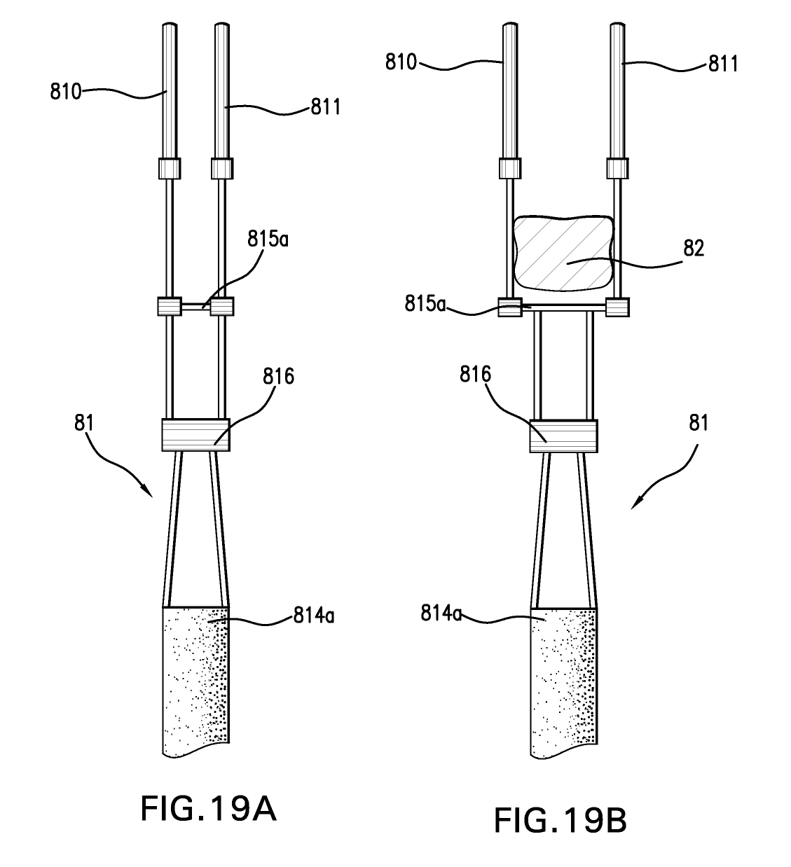

The distracting means may comprise any means suitable for distracting the left insertion fingers from the right insertion fingers. For example, in certain embodiments, the distracting means may comprise superior track 815a and inferior track 815b. In these embodiments, fingers 810 and 811 are slidably connected to track 815a such that fingers 810 and 811 are capable of sliding laterally from an undeployed position (show in FIG. 19A) to a deployed position (shown in 19B) using deployment block 82. In certain embodiments, deployment block 82 is slid into the insertion tool as shown in FIG. 19B to distract left fingers 810 and 812 from right fingers 811 and 813. In certain embodiments, the inferior track may further comprise a lip (not shown) on which the deployment block can rest, thus preventing the block from falling out of the insertion tool. The present invention also provides a kit for replacing a natural intervertebral disc with an extendable intervertebral disc prosthesis system including an extendable intervertebral disc prosthesis system and the insertion tool described in this paragraph.

A non-limiting example of a process for inserting an extendable intervertebral disc prosthesis system will now be described. The patient is placed in the prone position on a standard radiolucent operating table. In certain embodiments, the patient is placed in the supine position, and the approach may be anterior or lateral, for example. However, a key feature of the present invention is its ability to be inserted via a posterior approach, and in preferred embodiments the approach is posterior. In embodiments in which the approach is posterior, the patient is positioned accordingly. A patient's natural intervertebral disc is removed from its intervertebral disc space. A superior endplate assembly and an inferior endplate assembly each capable of assuming an unextended position and an extended position are provided. Both the superior and inferior endplate assemblies in the unextended position are simultaneously inserted in the excised intervertebral disc space. After being positioned in the intervertebral disc space, the endplate assemblies are simultaneously distracted to the extended position. An inner core is then implanted between the superior and inferior endplate assemblies.

In certain embodiments, a method of implanting an extendable intervertebral disc prosthesis may comprise additional steps. For example, in certain embodiments the method may further comprise the steps of engaging a set screw or other securing member to lock the inner core in place between the superior and inferior endplate assemblies. In these embodiments, the inner core is held in place between the two endplate assemblies, thus preventing it from slipping out of the applied position between the endplate assemblies. In addition, the insertion tool described above may be used with any of the methods described in the present invention.

Although an exemplary method has been described for implanting an extendable intervertebral disc prosthesis system, one of skill in the art will appreciate that the endplate assemblies, the telescoping prosthesis system, and other embodiments of the present invention may be implanted using similar methods that fall under the scope of the present invention.

While various embodiments have been described, other embodiments are plausible. It should be understood that the foregoing descriptions of various examples of an extendable intervertebral disc prosthesis system and components are not intended to be limiting, and any number of modifications, combinations, and alternatives of the examples may be employed to facilitate the effectiveness of an expandable disc prosthesis.

The examples described herein are merely illustrative, as numerous other embodiments may be implemented without departing from the spirit and scope of the exemplary embodiments of the present invention. Moreover, while certain features of the invention may be shown on only certain embodiments or configurations, these features may be exchanged, added, and removed from and between the various embodiments or configurations while remaining within the scope of the invention. Likewise, methods described and disclosed may also be performed in various sequences, with some or all of the disclosed steps being performed in a different order than described while still remaining within the spirit and scope of the present invention.

What is claimed is:

1. An extendable intervertebral disc prosthesis endplate assembly having a longitudinal axis, comprising:
a slotted plate, comprising:
a body having an outer surface, an inner surface, a posterior surface, and an anterior surface;
at least one slot defined by at least the inner surface of the body of the slotted plate;
an engagement plate, comprising:
a body having a first major surface, a second major surface, a posterior surface, an anterior surface, and a medial wall;
a lip extending from the medial wall of the body of the engagement plate, the lip having a contact surface;
at least one engagement member extending from the contact surface of the lip;
wherein, when the at least one engagement member is received by the at least one slot of the slotted plate and slides along the assembly's longitudinal axis within the at least one slot, the assembly moves from an unextended position having a first width to an extended position having a second width greater than the first width.

2. The endplate assembly of claim 1, further comprising:
an extending portion in fluid communication with the inner surface of the slotted plate, the engagement plate, or both, the extending portion located on either the slotted plate, the engagement plate, or both the slotted plate and the engagement plate; and
wherein, in an applied position of the assembly, the extending portion projects in the inferior direction.

3. The endplate assembly of claim 1, further comprising:
an extending portion in fluid communication with the inner surface of the slotted plate, the engagement plate, or both, the extending portion located on either the slotted plate, the engagement plate, or both the slotted plate and the engagement plate; and
wherein, in an applied position of the assembly, the extending portion projects in the superior direction.

4. The endplate assembly of claim 2, wherein the extending portion is an elbow defining a groove extending along at least a portion of the space between the posterior and anterior surfaces of the body of the slotted plate, the body of the engagement plate, or both.

5. The endplate assembly of claim 2, wherein the extending portion is a hook portion defining a groove extending along at least a portion of the space between the posterior and anterior surfaces of the body of the slotted plate, the body of the engagement plate, or both.

6. The endplate assembly of claim 2, wherein both the slotted plate and the engagement plate comprise an extending portion that projects from the inner surfaces of the slotted plate and the engagement plate in the inferior direction in an applied position of the assembly.

7. The endplate assembly of claim 3, wherein the extending portion is an elbow defining a groove extending along at least a portion of the space between the posterior and anterior surfaces of the body of the slotted plate, the body of the engagement plate, or both.

8. The endplate assembly of claim 3, wherein the extending portion is a hook portion defining a groove extending along at least a portion of the space between the posterior and anterior surfaces of the body of the slotted plate, the body of the engagement plate, or both.

9. The endplate assembly of claim 3, wherein both the slotted plate and the engagement plate comprise an extending portion that projects from the inner surfaces of the slotted plate and the engagement plate in the superior direction in an applied position of the assembly.

10. An extendable intervertebral disc prosthesis system, comprising:
(A) a superior endplate assembly;
(B) an inferior endplate assembly; and
(C) an inner core, comprising a core body;
wherein each endplate assembly has a longitudinal axis and comprises:
(i) a slotted plate, comprising:
(a) a body having an outer surface, an inner surface, a posterior surface, and an anterior surface;
(b) at least one slot defined by at least the inner surface of the body of the slotted plate;
(ii) an engagement plate, comprising:
(a) a body having an outer surface, an inner surface, a posterior surface, an anterior surface, and a medial wall;
(b) a lip extending from the medial wall of the body of the engagement plate, the lip having a contact surface;
(c) at least one engagement member extending from the contact surface of the lip;
wherein, when the at least one engagement member is received by the at least one slot of the slotted plate and slides along the assembly's longitudinal axis within the at least one slot, the assembly moves from an unextended position having a first width to an extended position having a second width greater than the first width;
wherein the slotted plate, the engagement plate, or both further comprises an extending portion in fluid communication with the inner surface of the slotted plate, the engagement plate, or both;
wherein the inner core's body is configured to be slidably received by the extending portions of the superior and inferior endplate assemblies; and
wherein, when the inner core is slid between the superior and inferior endplate assemblies, the superior and inferior endplate assemblies are held in the extended position.

11. An extendable intervertebral disc prosthesis system, comprising:
(A) a superior endplate assembly;
(B) an inferior endplate assembly; and
(C) an inner core comprising a core body having at least one projection extending from a surface of the core body;
wherein each endplate assembly has a longitudinal axis and comprises:
(i) a slotted plate, comprising:
(a) a body having an outer surface, an inner surface, a posterior surface, and an anterior surface;
(b) at least one slot defined by at least the inner surface of the slotted plate;
(c) a hook portion defining a groove extending along at least a portion of the space between the posterior and anterior surfaces of the body of the slotted plate;
(ii) an engagement plate, comprising:
(a) a body having an outer surface, an inner surface, a posterior surface, an anterior surface, and a medial wall;
(b) a lip extending from the medial wall of the body of the engagement plate, the lip having a contact surface;
(c) at least one engagement member extending from the contact surface of the lip; and
(d) a hook portion defining a groove extending along at least a portion of the space between the posterior and anterior surfaces of the body of the engagement plate;
wherein, when the at least one engagement member is received by the at least one slot of the slotted plate and slides along the assembly's longitudinal axis within the at least one slot, the assembly moves from an unextended position having a first width to an extended position having a second width greater than the first width;
wherein the at least one projection of the inner core is configured to be slidably received by the grooves of the slotted plates and the engagement plates; and
wherein, when the inner core is slid between the superior and inferior endplate assemblies, the superior and inferior endplate assemblies are held in the extended position and each endplate assembly is interlocked to the inner core.

12. The prosthesis system of claim 11, further comprising means for securing the prosthesis to adjacent superior or inferior vertebral bodies, the securing means located on the outer surface of the slotted plates, the engagement plates, or both.

13. The prosthesis system of claim 11, further comprising a plurality of serrations for securing the prosthesis to adjacent superior or inferior vertebral bodies, the plurality of serrations located on the outer surface of the slotted plates, the engagement plates, or both.

14. The prosthesis system of claim 11, further comprising at least one keel for securing the prosthesis to adjacent superior or inferior vertebral bodies, the at least one keel located on the outer surface of the slotted plates, the engagement plates, or both.

15. The prosthesis system of claim 11, further comprising at least one spiked protrusion for securing the prosthesis to adjacent superior or inferior vertebral bodies, the at least one spiked protrusion located on the outer surface of the slotted plates, the engagement plates, or both.

16. The prosthesis system of claim 11, wherein the at least one slot of the slotted plate is tapered such that the at least one slot's width decreases in the direction of the engagement plate in an applied position of the assembly.

17. The prosthesis system of claim 11, wherein at least one of the slotted plates or at least one of the engagement plates comprises a rigid material.

18. The prosthesis system of claim 11, wherein the at least one engagement member comprises means for securing the prosthesis to adjacent superior or inferior vertebral bodies.

19. The prosthesis system of claim 11, wherein the at least one engagement member has a mushroom shape.

20. The prosthesis system of claim 11, further comprising means for accepting an insertion tool located on a surface of each of the slotted plates and each of the engagement plates.

21. The prosthesis system of claim 20, wherein the means for accepting an insertion tool are located on the posterior surfaces of the slotted plates and engagement plates.

22. The prosthesis system of claim 20, wherein the means for accepting an insertion tool are located on the anterior surfaces of the slotted plates and the engagement plates.

23. The prosthesis system of claim 11, further comprising at least one recess configured to receive a distal end of an insertion tool.

24. The prosthesis system of claim 23, wherein the recesses comprise internal threads.

25. The prosthesis of claim 11, further comprising at least one protrusion configured to engage with a distal end of an insertion tool.

26. The prosthesis of claim 25, wherein the protrusions comprise external threads.

27. The prosthesis of claim 11, wherein the bodies of the slotted plates, the engagement plates, or both further comprise pores extending from the outer surface to the inner surface.

28. The prosthesis of claim 11, wherein the bodies of the slotted plates, the engagement plates, or both further comprise a porous layer coating the outer surfaces.

29. The prosthesis of claim 11, wherein the inner core is replaced by an interbody fusion cage, the interbody fusion cage comprising a cage body and at least one projection extending from a surface of the cage body;
wherein the cage's at least one projection is configured to be slidably received by the grooves of the slotted plates and the engagement plates; and
wherein, when the cage is slid between the superior and inferior endplate assemblies, the superior and inferior endplate assemblies are held in the extended position and each endplate assembly is interlocked to the cage.

30. The prosthesis of claim 11, further comprising means for preventing the inner core from shifting out from the proper position between the superior and inferior endplate assemblies.

31. A telescoping intervertebral disc prosthesis system, comprising:
(A) a superior endplate assembly having a longitudinal axis;
(B) an inferior endplate assembly having a longitudinal axis;
(C) an inner core, comprising a core body and at least one projection extending from a surface of the core body;
wherein each endplate assembly comprises:
(i) an inner member, comprising:
(a) a body having a cross-sectional profile, an outer surface, an inner surface, a posterior surface, and an anterior surface;
(b) a hook portion defining a groove extending along at least a portion of the space between the posterior surface of the body of the inner member and the anterior surface of the body of the inner member;
(ii) an outer member, comprising:
(a) a body having an outer surface, an inner surface, a posterior surface, and an anterior surface;
(b) a longitudinal channel defined by a space large enough to receive the cross-sectional profile of the inner member;
(c) a hook portion defining a groove extending along at least a portion of the space between the posterior surface of the outer member and the anterior surface of the body of the outer member;
wherein, when the inner member is slid within the longitudinal channel of the outer member, the assembly moves from an unextended position having a first width to an extended position having a second width greater than the first width;
wherein the at least one projection of the inner core is configured to be slidably received by at least one of the grooves; and
wherein, when the inner core is slid between the superior and inferior endplate assemblies, each endplate assembly is held in the extended position and interlocked to the inner core.

32. A method for surgically implanting an extendable intervertebral disc prosthesis in an intervertebral disc space, the method comprising the steps of:
removing a patient's natural intervertebral disc from an intervertebral disc space;
providing a superior endplate assembly and an inferior endplate assembly, wherein each endplate assembly is capable of assuming an unextended position and an extended position;
simultaneously implanting, in the unextended position, the superior and inferior endplate assemblies into the intervertebral disc space;
simultaneously distracting the superior and inferior endplate assemblies into the extended position; and
implanting an inner core between the superior and inferior endplate assemblies.

33. The method of claim 32, further comprising engaging means for preventing the inner core from shifting out from the proper position between the endplate assemblies, the means located on at least one of the endplate assemblies.

34. The method of claim 32, further comprising providing an insertion tool comprising a plurality of insertion fingers for receiving the superior and inferior endplate assemblies, wherein the plurality of insertion fingers comprises a left superior insertion finger, a right superior insertion finger, a left inferior insertion finger, and a right inferior insertion finger.

35. The method of claim 34, wherein simultaneously distracting the superior and inferior endplate assemblies into the extended position comprises inserting a deployment block to laterally distract the left insertion fingers from the right insertion fingers.

36. The method of claim 34, further comprising distracting the superior insertion fingers from the inferior insertion fingers in the superior-inferior direction to achieve a suitable height for the endplate assemblies.

37. A method for surgically implanting an extendable intervertebral disc prosthesis in an intervertebral disc space, the method comprising the steps of:
removing a patient's natural intervertebral disc from an intervertebral disc space;
providing a superior endplate assembly and an inferior endplate assembly, wherein each endplate assembly is capable of assuming an unextended position and an extended position;
implanting in the unextended position either one of the superior endplate assembly or the inferior endplate assembly in the excised disc space;
distracting the either one of the superior endplate assembly or the inferior endplate assembly to an extended position;
implanting in the unextended position the other of the superior endplate assembly or the inferior endplate assembly in the excised disc space;
distracting the other of the superior endplate assembly or the inferior endplate assembly to an extended position; and
implanting an inner core between the superior and inferior endplate assemblies.

38. A kit for replacing a natural intervertebral disc with an extendable intervertebral disc prosthesis system, comprising:
the extendable intervertebral disc prosthesis system of claim 11; and
an insertion tool, comprising:
a plurality of insertion fingers, comprising:
a left superior insertion finger;
a right superior insertion finger;
a left inferior insertion finger; and
a right inferior insertion finger;
means for laterally distracting the left insertion fingers from the right insertion fingers; and
a pair of handles pivotally connected to the means for laterally distracting, wherein the handles are capable of distracting, in the superior-inferior direction, the superior insertion fingers from the inferior insertion fingers;
wherein the plurality of fingers is connected either directly or indirectly to the means for laterally distracting.

39. The kit of claim 38, wherein the insertion tool further comprises means for attaching to the superior and inferior endplate assemblies of the extendable intervertebral disc prosthesis system, the means located on a distal end of each of the plurality of insertion fingers.

40. A kit for replacing a natural intervertebral disc with an extendable intervertebral disc prosthesis system, comprising:
the extendable intervertebral disc prosthesis system of claim 11; and:
an insertion tool, comprising:
a superior track;
an inferior track;
a left superior insertion finger;
a right superior insertion finger;
wherein the superior insertion fingers are slidably connected to the superior track such that the superior insertion fingers are capable of moving laterally along the superior track;
a left inferior insertion finger;
a right inferior insertion finger;
wherein the inferior insertion fingers are slidably connected to the inferior track such that the inferior insertion fingers are capable of moving laterally along the inferior track; and
a pair of handles pivotally connected to the superior and inferior tracks, wherein the handles are capable of distracting, in the superior-inferior direction, the superior insertion fingers from the inferior insertion fingers.

41. The kit of claim 40, wherein the inferior track of the insertion tool further comprises a lip for receiving a deployment block.

42. The kit of claim 40, wherein the insertion tool further comprises means for attaching to the superior and inferior endplate assemblies of the extendable intervertebral disc prosthesis system, the means located on a distal end of each of the plurality of insertion fingers.
